(19) United States
(12) Patent Application Publication
(10) Pub. No.: US 2019/0381169 A1
(43) Pub. Date: Dec. 19, 2019

(54) PHARMACEUTICAL COMBINATIONS FOR IMMUNOTHERAPY

(71) Applicant: Technische Universitat Dresden, Dresden (DE)

(72) Inventors: Sebastian Brenner, Dresden (DE); Martin Ryser, Ixelles (BE); Cornelia Richter, Dresden (DE); Sebastian Thieme, Dresden (DE)

(21) Appl. No.: 16/393,214

(22) Filed: Apr. 24, 2019

Related U.S. Application Data
(63) Continuation of application No. 15/313,687, filed on Nov. 23, 2016, now abandoned.

(30) Foreign Application Priority Data
May 28, 2014 (EP) 14 170 362.9

(51) Int. Cl. A61K 39/39; G01N 33/50; A61K 39/35; A61K 49/00

(52) U.S. Cl. CPC A61K 39/39; G01N 33/5047; A61K 39/35; A61K 2039/55516; G01N 33/5041; G01N 33/505; G01N 2333/71; A61K 49/0008

(57) ABSTRACT

The present invention relates generally to a method for regulating immune reactions and test substances useful for same. Specifically, the method of the present invention relates to the modulation of the nerve growth factor receptor p75^{NTR}, which is expressed by plasmacytoid dendritic cells. More specifically, the invention relates to a combination comprising at least one modulator of p75^{NTR} signalling selected from a p75^{NTR} antagonist or p75^{NTR} agonist and at least one TLR receptor agonist selected from an agonist of TLR7 and/or TLR9. The invention further relates to the use of a combination of antagonists and agonists of p75^{NTR} signalling and agonists of TLR7 and/or TLR9 as vaccine adjuvants and the invention provides vaccine compositions comprising antagonists and agonists of p75^{NTR} signalling and agonists of TLR7 and/or TLR9. The agonists and antagonists of p75^{NTR} signalling are useful in the manufacture of drugs for controlling cytokine function, antigen presentation, activation and proliferation of lymphocytes, which is important for the treatment of a range of conditions including cancer, inflammatory conditions, immunological disorders, growth disorders, infections and any other conditions involving p75^{NTR} signal transduction. The invention provides assays to screen for a range of agonists and antagonists of p75^{NTR} useful in modulating cytokine function, activation and proliferation of lymphocytes. The present invention further provides, therefore, screening assays for agonists and antagonists of p75^{NTR}-modulated immune responses.

### CROSS-REFERENCE TO RELATED APPLICATIONS

This application is a continuation of U.S. patent application Ser. No. 15/313,687, filed on Nov. 23, 2016, which is a 371 National Stage of International Patent Application No. PCT/EP2015/061851, filed on May 28, 2015, which claims priority of European Patent Application No. 14 170 362.9, filed on May 28, 2014, each of which is incorporated herein by reference.

### FIELD OF THE INVENTION

The present invention relates generally to a method for regulating immune reactions and test substances useful for same. Specifically, the method of the present invention relates to the modulation of the nerve growth factor receptor p75^{NTR}, which is expressed by human and murine plasmacytoid dendritic cells (PDC). More specifically, the invention relates to a combination comprising at least one modulator of p75^{NTR} signalling selected from a p75^{NTR} antagonist or p75^{NTR} agonist and at least one TLR receptor agonist selected from an agonist of TLR7 and/or TLR9 that can be used for treating a subject suffering from a disease or pathological condition that involves p75^{NTR} signalling or as a vaccine adjuvant. The invention provides assays to screen for a range of agonists and antagonists useful in modulating cytokine function and antigen presentation by PDC, and the activation and proliferation of lymphoid and myeloid cells, e.g. T-cells. The present invention further provides, therefore, screening assays for agonists and antagonists of p75^{NTR}-modulated immune responses. Such agonists and antagonists are useful in the manufacture of vaccine compositions or drugs for controlling cytokine function, antigen presentation, activation and proliferation of lymphoid and myeloid cells, which is important for the prevention or treatment of a range of conditions including infections, cancer, inflammatory reactions, immunological disorders, growth disorders and any other conditions involving p75^{NTR} signal transduction.

### SEQUENCE LISTING INCORPORATION

Biological sequence information for this application is included in an ASCII text file, filed with the application, having the file name “MAI207CON1_SEQ.txt”, created on Apr. 24, 2019, and having a file size of 11,173 bytes, which is incorporated herein by reference.

### BACKGROUND OF THE INVENTION

The immune system functions to protect individuals from infective agents, e.g., bacteria, multi-cellular organisms, and viruses, as well as from cancers. This system includes several types of lymphoid and myeloid cells such as T-cells, B-cells, monocytes, macrophages, dendritic cells (DCs), eosinophils and neutrophils. These lymphoid and myeloid cells often produce signalling proteins known as cytokines. The immune response includes inflammation, i.e., the accumulation of immune cells systemically or in a particular location of the body and can lead to autoimmune disease or Graft-versus-Host disease (GvHD). In response to an infective agent or foreign substance, immune cells secrete cytokines which, in turn, modulate immune cell proliferation, development, differentiation, migration or activation. Cytokines have been implicated in the pathology of a number of disorders and conditions.

In more detail, the human immune system has developed to give us protection against microbes by coordination of innate (non-specific) and adaptive/acquired immune mechanisms (combination of cell mediated and humoral immune responses). The innate immunity cells include phagocytes (macrophages, other DCs, neutrophils), mast-cells, basophils and eosinophils, innate T-cells (γδT-cells), epithelial cells, NK (natural killer) cells and PDC. These cells function as first line of body defence against any attacking microbes by secreting anti-microbial cytokines e.g. on viral encounter PDC secret type I interferons (IFN), a family of cytokines with potent anti-viral activity. Adaptive immunity, on the other hand, includes T helper cells (Th1, Th2 and Th17) and cytotoxic T-cells (CTL) based immune responses (cell mediated immunity) and B-cells that differentiate into antigen specific antibody producing B plasma cells (humoral immunity). PDC, in addition to their vital role in innate immunity, have the ability to trigger T-cell responses and regulate B-cell growth and differentiation into antibody secreting plasma cells. PDC contribute essentially in regulating and bridging antigen induced innate and adaptive immune responses.

PDC express endosomal toll like receptors 7 (TLR7) and 9 (TLR9) that are able to bind single stranded viral RNA and bacterial or viral DNA, respectively. Upon activation of TLR7 or TLR9, a signalling cascade is activated involving e.g. MyD88, TRAF6, IRAK4, IRF3 and IRF7, which ultimately leads to the production of very high levels of interferon alpha (IFNα). IFNα induces Th1 and CTL immune reactions and has multiple functions in the human body in viral defence, in the elimination of tumour cells, but also in the induction of autoimmunity. For a long time interferon production upon toll like receptor activation associated with the induction of a Th1 immune reaction seemed to be the only function that could be attributed to PDCs.

TRAF3 and TRAF6 are human protein members of the TNF receptor associated factor (TRAF) protein family. TRAF proteins are associated with, and mediate signal transduction from members of the TNF receptor superfamily. These proteins mediate the signalling not only from the members of the TNF receptor superfamily, but also from the members of the Toll/IL-1 family.

Loss of Myeloid Differentiation primary response gene 88 (MyD88) expression is associated with decreased resistance to bacterial infections. Moreover, mutated forms of MyD88 have been identified in various human lymphomas (Hawn et al., J Infect Dis. (2006) 193 (12): 1693-1702).

Interferon regulatory factor 3 (IRF3) and 7 (IRF7) are members of the interferon regulatory factor family of transcription factors. IRF3 and IRF7 have been shown to play a role in the transcriptional activation of virus-inducible cellular genes, including pro-inflammatory and type I interferon genes. In particular, IRF7 regulates many IFNα genes. Constitutive expression of IRF7 is largely restricted to lymphoid tissue; particularly PDCs. Expression of IRF7 is, however, inducible in many tissues.

Neurotrophins are the family of proteins which are considered to have an essential role in the development of the vertebrate nervous system. Nerve growth factor (NGF) is the best characterized member of the neurotrophin family and was the first to be isolated. Other members of the ever growing family of neurotrophins include: Brain derived nerve factor (BDNF), Neurotrophin-3 (NT-3) and Neurotrophin-4 and 5 (NT-4 and NT-5). Neurotrophins mediate their effects by binding to two different receptors classes with different affinities: i) high affinity nerve growth factor receptor which includes: the Trk A, Trk B and Trk C (tropomyosin-receptor kinase A, B and C), and ii) low affinity nerve growth factor receptor (LNGFR), member of the tumour necrosis factor receptor superfamily, which is also known as p75^{NTR} or CD271 (Lykissas et al., Curr Neurovasc Res. 2007 May; 4(2): 143-51).

In recent years it has been demonstrated that PDCs also play a pivotal role in the regulation of immune responses to exogenous antigens and self-antigens. It could be demonstrated that depletion of PDCs in mice aggravates allergic asthma, which is a Th2 immune response, but also worsens the autoimmune reaction in experimental autoimmune encephalomyelitis (EAE), a mouse model for multiple sclerosis, which is based on a Th1 immune response. From those results it could be deducted that PDCs have a major regulatory function to induce tolerance, but might also be involved in the escape of tumour cells from host immunity.

The induction of immune reaction and the inhibition of tolerance are major determinants for the success of vaccination strategies. Classical vaccines rely on the induction of Th2 immune reactions to induce humoral immunity against the vaccine antigens. As attenuated vaccines do not induce a strong immune reaction, adjuvants are used to potentiate the immune response. The most common Th2 inducing adjuvants are aluminium salts. In order to kill intracellular organisms or to eliminate tumour cells, Th1 and CTL immune responses need to be induced, for which therefore different adjuvants are to be used. Most of the Th1 inducing adjuvants act via activation of TLRs. An overview on current adjuvants or new adjuvants that are being evaluated in clinical trials are shown in table 1 below:

TABLE 1
Adjuvants and new adjuvants that are currently evaluated in clinical trials

| | | | | Clinical phase or |
|---|---|---|---|---|
| | | Mechanism or | Type of immune | licensed product |
| Adjuvant name | Class | receptor | response | name |
| dsRNA analogues (for | IM | TLR3 | Ab, Th1, CD8+ T-cells | Phase 1 |
| example, poly(I:C)) | | | | |
| Lipid A analogues (for | IM | TLR4 | Ab, Th1 | Cervarix, Supervax, |
| example, MPL, RC529, | | | | Pollinex Quattro, |
| GLA, E6020) | | | | Melacine |
| Flagellin | IM | TLR5 | Ab, Th1, Th2 | Phase 1 |
| Imidazoquinolines (for | IM | TLR7 and TLR8 | Ab, Th1 | Aldara |
| example, Imiquimod, R848) | | | | |
| CpG ODN | IM | TLR9 | Ab, Th1, CD8+ T-cells | Phase 3 |
| Saponins (for example, QS21) | IM | Unknown | Ab, Th1, Th2, CD8+ | Phase 3 |
| | | | T-cells | |
| C-type lectin ligands (for | IM | Mincle, Nalp3 | Ab, Th1, Th17 | Phase 1 |
| example, TDB) | | | | |
| CD1d ligands (for example, | IM | CD1d | Ab, Th1, Th2, CD8+ | Phase 1 |
| α- galactosylceramide) | | | NKT-cells | |
| Aluminium salts (for | PF | Nalp3, ITAM, | Ab, Th2 | Numerous licensed |
| example, aluminium | | Ag delivery | | products |
| oxyhydroxide, aluminium | | | | |
| phosphate) | | | | |
| Emulsions (for example, | PF | Immune cell | Ab, Th1, Th2 | Fluad, Pandemrix |
| MF59, AS03, AF03, SE) | | recruitment, | | |
| | | ASC, Ag | | |
| | | uptake | | |
| Virosomes | PF | Ag delivery | Ab, Th1, Th2 | Epaxal, Inflexal V |
| AS01 (MPL, QS21, | C | TLR4 | Ab, Th1, CD8+ T-cells | Phase 3 |
| liposomes) | | | | |
| AS02 (MPL, QS21, emulsion) | C | TLR4 | Ab, Th1 | Phase 3 |
| AS04 (MPL, aluminium salt) | C | TLR4 | Ab, Th1 | Cervarix |
| AS15 (MPL, QS21, CpG, | C | TLR4 and TLR9 | Ab, Th1, CD8+ T-cells | Phase 3 |
| liposomes) | | | | |
| GLA-SE (GLA, emulsion) | C | TLR4 | Ab, Th1 | Phase 1 |
| IC31 (CpG, cationic peptide) | C | TLR9 | Ab, Th1, Th2, CD8+ | Phase 1 |
| | | | T-cells | |
| CAF01 (TDB, cationic | C | Mincle, Ag | Ab, Th1, CD8+ T-cells | Phase 1 |
| liposomes) | | delivery | | |
| ISCOMs (saponin, | C | Unknown | Ab, Th1, Th2, CD8+ | Phase 2 |
| phospholipid) | | | T-cells | |

Ab, antibody; Ag, antigen; ASC, apoptosis-associated speck-like protein containing caspase recruitment domain; C, combination of immunomodulatory molecule and particulate formulation; dsRNA, double-stranded RNA; IM, immunomodulatory molecule; ITAM, immunoreceptor tyrosine-based activation motif; PF, particulate formulation; TDB, trehalose dibehenate. Some particulate formulations (such as aluminium salts and emulsions) also generate immunomodulatory activity.

WO 2012/101664 concerns the use of at least one p75^{NTR} receptor inhibitor, alone or in association with at least one TrkA receptor activator, or at least one TrkA receptor activator, for the treatment of chronic inflammatory diseases, for the treatment of chronic inflammatory diseases as, for example, rheumatoid arthritis, juvenile idiopathic arthritis, psoriasis, multiple sclerosis, intestinal chronic inflammatory diseases, Lupus Erythematosus.

WO 97/37228 relates to methods for evaluating the risk of an individual to develop Alzheimer's disease using cultured neural crest-derived melanocytes. Also described are methods of therapy for Alzheimer's disease using peptides that bind to the neurotrophin receptor (p75^{NTR}) and competitively inhibit the binding of β-amyloid to the p75^{NTR}.

US 2008/064036 provides a method to identify a test compounds capability to modulate p75^{NTR} induced apoptosis, said method comprising: i.) Transfecting a suspension of eukaryotic cells with a vector encoding p75^{NTR} (SEQ ID No.2) or a cell death inducing fragment thereof, ii.) Contacting said cells with the compound to be tested, and iii.) Determine the apoptotic response in said cells, wherein an alteration in apoptotic response in the presence of said test compound compared to the apoptotic response in the absence of the test compound is an indication of the ability of the test compound to modulate p75^{NTR} induced apoptosis.

### SUMMARY OF THE INVENTION

The invention is based on the unexpected finding that plasmacytoid dendritic cells (PDC) express the nerve growth factor receptor p75^{NTR}. Based on broad evidence, generated in in vitro experiments and various mouse models, it could further be established that p75^{NTR} is an important regulator of PDC driven immune responses, where p75^{NTR} activation on TLR7 or TLR9 activated PDCs inhibits CTL and Th1 responses and directs the immune response more to a Th2 response, as shown in cytokine secretion assays and cell proliferation assays, and mouse disease models of CTL, Th1 and Th2, e.g., allergic asthma, GvHD and autoimmune type I diabetes.

The invention therefore provides a method of modulating an activity of a cell that comprises contacting the cell with an agonist or antagonist of p75^{NTR}, where the cell expresses TLR7 and/or TLR9 and p75^{NTR}, wherein the p75^{NTR} agonist or antagonist modulates an immune response and/or cell proliferation in response to agonists of TLR7 or TLR9.

Also provided is the above method wherein the cell is preferably a PDC isolated from primary tissue or generated by differentiation from primary tissue in vitro, or a cell line derived from primary PDCs or in vitro differentiated primary tissue.

In another aspect, the invention provides the use of a pharmaceutical combination of an agonist TLR7 or TLR9 and an agonist or antagonist of p75^{NTR} for treating a subject suffering from a disease or pathological condition that involves p75^{NTR} signalling, such as an infection, inflammatory disorder, immune disorder or cancer, wherein the disease or pathological condition is mediated by monocytes or macrophages, neutrophils, T-cells or B-cells, DCs, epithelial cells or endothelial cells. In a further embodiment, the disease is mediated via PDCs.

P75^{NTR} on PDCs functions as a master switch in the regulation of PDC mediated immune responses. The modulation of immune responses is the major function of vaccine adjuvants. Therefore agonist and antagonists of p75^{NTR} in combination with PDC activators, preferably agonists of TLR7 and/or TLR9 provide a means for novel adjuvants. The invention therefore further provides vaccine compositions comprising an agonist or antagonist of p75^{NTR} signalling.

Activation of p75^{NTR} on activated PDCs strongly induces Th2 immune responses. Therefore agonists can boost immunization responses in Th2 dependent vaccines. The directed immune response is similar to aluminium salts but not related to an induction of local inflammation.

P75^{NTR} agonists might be used to replace current vaccine adjuvant components or could be used in combination to further boost a vaccine response.

In another embodiment the invention relates to the use of a vaccine composition comprising a p75^{NTR} agonist for modulating immune responses comprising but not limited to stimulation of Th2 immune responses, suppression of Th1 immune responses, suppression of Th17 immune responses, suppression of CTL responses and suppression of regulatory T-cell induced tolerance and the like.

In yet another embodiment the invention relates to the use of a vaccine composition comprising a p75^{NTR} antagonist for modulating immune responses comprising but not limited to suppression of Th2 immune responses, stimulation of Th1 immune responses, stimulation of Th17 immune responses, stimulation of CTL responses and stimulation of regulatory T-cell induced tolerance and the like.

These combinations of activators of PDCs with agonists or antagonists of p75^{NTR} signalling can be incorporated into pharmaceutical compositions, preferably in vaccine compositions, for use in immunotherapy.

Another embodiment of the present invention provides a method of screening for a compound that modulates p75^{NTR} signalling on a eukaryotic cell that co-expresses p75^{NTR} and at least one of the toll like receptors TLR7 or TLR9.

Another embodiment of the present invention provides a method of screening for a compound that modulates p75^{NTR} signalling on a eukaryotic cell with a p75^{NTR} knockout, or a reduced expression of p75^{NTR}, or expressing a non-functional p75^{NTR} variant, and at least one of the toll like receptors TLR7 or TLR9.

Another embodiment provides a method comprising contacting a candidate compound to a mouse with p75^{NTR} knockout, or with a reduced p75^{NTR} expression, or expressing a non-functional p75^{NTR} variant, and determining the physiological activity in the contacted p75^{NTR} knockout mouse; determining the physiological activity in a mouse with p75^{NTR} knockout, or with a reduced p75^{NTR} expression, or expressing a non-functional p75^{NTR} variant, not contacted with the candidate compound; and comparing the physiological activities of the contacted mouse with a with p75^{NTR} knockout, or with a reduced p75^{NTR} expression, or expressing a non-functional p75^{NTR} variant, and the non-contacted mouse with p75^{NTR} knockout, or with a reduced p75^{NTR} expression, or expressing a non-functional p75^{NTR} variant, as well as the above method wherein the physiological activity comprises an immune activity; inflammation, hyperreactivity, or a proliferative activity.

### BRIEF DESCRIPTION OF THE DRAWINGS

FIGS. 1A and 1B show the effect of NGF on murine PDCs during allergen-mediated immune response. In the bronchoalveolar lavage fluid (BALF), numbers of eosinophils and lymphocytes were significantly augmented when the OVA up-take by PDCs was carried out in the presence of NGF compared to PDCs incubated with OVA alone, whereas number of macrophages decreased.

FIG. 1C shows that OVA-loaded PDCs treated with NGF caused increased production of Th2 cytokines (IL-4, IL-5 and IL-13) in the lung in comparison to PDCs pulsed with OVA in the absence of NGF.

FIG. 1D shows histological lung sections from mice that received OVA-loaded PDCs showed increased perivascular inflammation and enhanced mucus production. Treatment of PDCs with NGF during OVA-uptake potentiated the inflammatory phenotype in the lung.

FIGS. 2 A and 2B show the results of the investigation of the role of p75^{NTR} expressed on murine PDCs in the process of disease triggering in a mouse model of OVA-mediated allergic asthma. After provocation with OVA aerosol characteristic symptoms of asthma like severe eosinophilia, lung inflammation and intensive mucus production were analyzed. p75^{NTR+/+} mice (wildtype) and p75^{NTR-/-} mice (knockout) treated with OVA-loaded p75^{NTR-/-} PDCs showed significantly reduced numbers of immune cells in the BALF (lymphocytes and eosinophils) compared to mice that received p75^{NTR+/+} PDCs.

FIG. 2C shows that OVA-mediated immune response further lead to increased Th2 cytokine secretion (IL-4, IL-5 and IL-13) in the BALF of mice treated with p75^{NTR+/+} PDCs but not in mice that received p75^{NTR-/-} PDCs.

FIGS. 2D and 2E show that perivascular inflammation and Goblet-cell hyperplasia in the lung were diminished in mice treated with p75^{NTR-/-} PDCs compared to mice treated with p75^{NTR+/+} PDCs.

FIGS. 3A and 3B show the results of the investigation of the role of p75^{NTR} expressed on murine PDCs in the process of CPG oligodeoxynucleotide stimulated immune response in vitro. Murine PDCs from the p75^{NTR+/+} (wildtype) strain express the low affinity neurotrophin receptor p75^{NTR}, whereas the p75^{NTR-/-} (knockout) strain does not. The p75^{NTR+/+} PDCs do not express any of the other neurotrophin Trk receptors (FIG. 3A; with antibody staining: continuous line; without antibody staining: hatched area).

FIG. 3C shows that in contrast to p75^{NTR-/-} PDCs, CPG A induced IFNα secretion of p75^{NTR+/+} PDCs was reduced upon addition of NGF in a concentration dependent manner, illustration a reduction of Th1 response.

FIG. 3D shows that p75^{NTR+/+} PDCs secreted significantly higher amounts of pro-inflammatory cytokines IL-6 and TNFα after stimulation with the Th2 inducing oligodeoxynucleotide CPG B.

FIG. 3E shows that also expression of the Toll-like receptor TLR9 expressed on PDCs was negatively influenced by NGF addition to CPG A stimulated p75^{NTR+/+} PDCs, whereas p75^{NTR-/-} showed now difference in TLR9 expression.

FIG. 3F shows that addition of NGF to Th1-response inducing oligodeoxynucleotide CPG A stimulated p75^{NTR+/+} PDCs react with a reduced expression of MyD88 and TRAF6, and a reduced activation (phosphorylation) of the signalling proteins IRF-3, IRF7, IKK and c-Jun.

FIG. 3G shows that co-Incubation of p75^{NTR+/+} PDCs with pro-inflammatory, Th2-response inducing oligodeoxynucleotide CPG B and NGF induced increased expression of MyD88 and TRAF3. Also activation (phosphorylation) of the signalling proteins IRF3, IRF7, IKK and c-Jun was increased.

FIG. 4A shows the effect of NGF at the expression of Major Histocompatibility Complex proteins of Class I (MHC class I proteins) and/or of Class II (MHC Class II proteins) on murine PDCs co-stimulated with Toll-like receptor ligands CPG A and B. p75^{NTR+/+} (wildtype) PDCs react with an decreased expression of MHCII after addition of NGF to culture containing the Th1-response inducing CPG A (without NGF: continuous line, with NGF: dashed line).

FIG. 4B shows that PDCs stimulated with Th2-response inducing CPG B showed further increase in MHCII expression upon addition of NGF to the culture (without NGF: continuous line, with NGF: dashed line).

FIG. 4C shows that compared to p75^{NTR-/-} (knockout) PDCs, addition of NGF to p75^{NTR+/+} PDCs lead to a further increased expression of MHCI induced by pro-inflammatory CPG B (without NGF: continuous line, with NGF: dashed line). PDCs without staining are depicted as hatched area histogram.

FIGS. 5A and 5B show the influence of NGF on the secretion of the T-cell secreted Th1 cytokines IFNγ and IL-2 in a co-culture of murine PDCs and T-cells. PDCs were isolated from either p75^{NTR+/+} (wildtype) or p75^{NTR-/-} (knockout) mouse strain. T-cells were isolated from OTII mouse strain expressing ovalbumin peptide specific T-cell receptors. In the presence of p75^{NTR+/+} PDCs presenting the ovalbumin peptide (OVA) to the T-cells, T-cells secrete the Th1 cytokines IFNγ (FIG. 5A) and IL-2 (FIG. 5B). Compared to co-culture with p75^{NTR-/-} PDCs, T-cells co-cultured with PDCs from the p75^{NTR+/+} strain react with reduced secretion of both Th1 cytokines upon addition of NGF.

FIG. 6A shows graphic representations of IFNα (pg/ml) produced by human PDC activated by, ODN 2216 (▴) vs. ODN 2216+NGF at 200 ng/ml (▪). IFNα secreted in supernatant by activated PDC was determined by ELISA. Data shown are the mean plus minus SEM (n=20). Level of significance was chosen p<0.05. Significant differences indicated by (p=0.0031) and ** as determined by student's paired t-test (two-tailed).

FIG. 6B shows that in addition, blocking of p75^{NTR} receptor by synthetic peptide PEP5 in the presence of NGF resulted in significantly increased secretion of IFNα. Level of significance indicated by *, and * * was determined by student's paired T-test (two tailed); ns=non-significant.

FIG. 7A shows the influence of NGF on the proliferation of T-cells and the secretion of pro-inflammatory cytokines in a co-culture of T-cells and PDCs isolated from allergic patients. Upon addition of NGF to the co-culture, T-cells showed an increased proliferation in the presence of specific allergen.

FIG. 7B shows that T-cells also react with an increasing secretion of pro-inflammatory cytokines IL-2 and IL-5. Values are shown as mean with SEM of four different allergic donors (n=4). Values were compared using one-way ANOVA multiple comparison method (Tukey's). Differences were considered significant when p<0.05.

Ag.: Allergen

FIG. 8A shows the results of the investigation of the role of p75^{NTR} expressed on murine PDCs in the process of CpG oligodeoxynucleotide stimulated immune response in vitro. Murine PDCs from the p75^{NTR+/+} (wildtype) strain express the low affinity neurotrophin receptor p75^{NTR} whereas the p75^{NTR-/-} (knockout) strain does not. In the absence of NGF, both, the p75^{NTR+/+} (wildtype) PDCs and p75^{NTR-/-} (knockout) PDCs display the same percentage of TLR9 expressing cells upon stimulation with CPG oligodeoxynucleotide type A (CpG A) or type B (CpG B), lipopolysaccharides (LPS) or Ovalbumin (OVA; FIG. 8A).

FIGS. 8B and 8C show that in contrast to p75^{NTR-/-} PDCs, p75^{NTR+/+} PDCs showed higher basal TLR9 expression level with or without stimulation either with CpG A (FIG. 8B) or CpG B (FIG. 8C). CpG-induced increase in TLR9 expression level was significantly decreased in the presence of NGF.

FIGS. 9A, 9B, 9C, 9D and 9E show the effect of NGF at the expression of Major Histocompatibility Complex proteins of Class II (MHC II; FIG. 9A) or of Class I (MHC I; FIG. 9C), as well as of co-stimulatory molecules ICOS-L (FIG. 9B), PD-L1 (FIG. 9D) and Ox40-L (FIG. 9E) on murine PDCs co-stimulated with Ovalbumin protein (OVA). p75^{NTR+/+} (wildtype) PDCs react with an increased expression of MHCII and ICOS-L after addition of NGF to culture containing the OVA. Compared to p75^{NTR-/-} (knockout) PDCs, addition of NGF to p75^{NTR+/+} PDCs lead to a decreased expression of MHCI, PD-L1 and Ox40L after addition of NGF.

FIGS. 10A and 10B show the influence of NGF on T-cells with regard to proliferation and cytokine secretion (IFNγ, IL-6 and TNFα) of T-cells in a co-culture with murine PDCs. PDCs were isolated from either p75^{NTR+/+} (wildtype) or p75^{NTR-/-} (knockout) mouse strain. T-cells were isolated either from OT-II mouse strain expressing ovalbumin peptide specific T-cell receptors on CD4+ T-cells (FIG. 10A) or from OT-I mouse strain expressing ovalbumin peptide specific T-cell receptors on CD8+ T-cells (FIG. 10B). In the presence of p75^{NTR+/+} PDCs presenting the ovalbumin protein to the T-cells, which in turn secrete the cytokines and proliferate. Compared to co-culture with p75^{NTR-/-} PDCs, CD4+ T-cells from OT-II strain co-cultured with PDCs from the p75^{NTR+/+} strain react with increased cytokine secretion and proliferation upon addition of NGF, whereas CD8+ T-cells from OT-I strain secreted less cytokines and showed reduced proliferation when NGF was present in co-culture.

FIG. 11 shows graphic representations of IL-6 (pg/ml) produced by human PDC activated by an FcεRIα-specific, IgE-crosslinking antibody in the presence of NGF with or without additional blocking of p75^{NTR} receptor by synthetic peptide PEP5. Values are normalized to antibody treatment only. IL-6 secreted by activated PDC was determined by ELISA. Data shown are the mean plus minus SEM (n=8). Blocking of p75^{NTR} receptor by synthetic peptide PEP5 in the presence of NGF resulted in significantly decreased secretion of IL-6.

FIGS. 12A and 12B show the effect of p75^{NTR} receptor blocking on murine PDCs during allergen-mediated immune response in the presence of NGF. In the bronchoalveolar lavage fluid (BALF), numbers of eosinophils (FIG. 12A) significantly decreased when the OVA up-take by PDCs was carried out in the presence of an p75^{NTR}-specific, blocking antibody compared to PDCs incubated with OVA and NGF alone, whereas number of macrophages increased (FIG. 12B).

FIGS. 12C and 12D show that OVA-loaded PDCs treated with blocking antibody caused decreased production of IL-4 and IL-5 in the lung in comparison to PDCs pulsed with OVA and NGF in the absence of p75^{NTR}-blocking antibody.

FIGS. 13A and 13B show the effect of NGF on the cumulative Graft-versus-Host disease (GvHD) incidence (FIG. 13A) and survival (FIG. 13B) in a Th2 prone xenotransplantation model. NSG mice transplanted with human, autologous T-cells and PDCs develop GvHD. When PDCs were cultured prior transplantation in the presence of NGF GvHD severity increased accompanied with increased mortality. Skipping of pre-stimulation of PDCs with CpG B abolished the accelerating NGF effect arguing for a TLR7/9 dependent process (data not shown).

FIG. 14 shows the effect of NGF on the development of diabetes in a Th1 prone type I diabetes model. RIP-CD80× RIP-LCMV-GP mice transplanted with LCMV-GP peptide stimulated PDCs develop autoimmune diabetes diagnosed by increased blood glucose level. When pre-stimulation of PDCs was done in the presence of NGF diabetes free time was significantly prolonged.

### DETAILED DESCRIPTION OF THE INVENTION

### Definitions

The term “p75^{NTR}” herein refers to the Low-Affinity Nerve Growth Factor Receptor (also called LNGFR, p75 neurotrophin receptor, TNFRSF16 (TNFR superfamily, Member 16), Gp80-LNGFR, p75, p75ICD, Member 16, CD271 or NGF receptor). “p75^{NTR}” is one of the two receptor types for the neurotrophins, a family of protein growth factors that stimulate neuronal cells to survive and differentiate. “p75^{NTR}”, as used herein shall embrace the p75^{NTR} protein as usually expressed in mammalian cells but also all splice variants thereof. Splice variants of p75^{NTR} can be formed by “alternative splicing”, a regulated process during gene expression that results in a single gene coding for multiple proteins. During the process of alternative splicing, particular exons of a gene may be included within or excluded from the finally processed messenger RNA (mRNA), which is produced from that gene. Consequently the proteins translated from alternatively spliced mRNAs will contain differences in their amino acid sequence and, often, in their structure. Preferably, in accordance with the present invention, the p75^{NTR} protein is encoded by the gene having the nucleic acid sequence of SEQ ID No. 4 (Gene ID 4804; NCBI reference sequence NM_002507.3). Most preferably, the p75^{NTR} protein as used herein has the amino acid sequence of SEQ ID No. 3 (Swiss-Prot Accession No. P08138.1).

“Activation,” “stimulation,” and “treatment,” as it applies to cells or to receptors, may have the same meaning, e.g., activation, stimulation, or treatment of a cell or receptor with a ligand, agonist or antagonist unless indicated otherwise by the context or explicitly.

“Activation” can refer to cell activation as regulated by internal mechanisms as well as by external or environmental factors.

“Ligand” encompasses natural and synthetic (artificial) ligands, e.g., cytokines, cytokine variants, analogues, muteins, and binding compositions derived from antibodies. “Ligand” also encompasses small molecules, e.g., peptide mimetics of cytokines, peptide mimetics of antibodies, nucleic acids and nucleic acid mimetics.

An “agonist” is a chemical, agent or ligand that binds to a receptor and activates the receptor to produce a biological response. Whereas an agonist causes an action, an antagonist blocks the action of the agonist and an inverse agonist causes an action opposite to that of the agonist.

A “p75^{N}R agonist” is a chemical, agent or ligand that binds to and activates the p75^{NTR}.

A “TLR7 agonist” is a chemical, agent or ligand that binds to and activates the toll-like receptor 7.

A “TLR9 agonist” is a chemical, agent or ligand that binds to and activates the toll-like receptor 9.

An “antagonist” is a ligand that blocks agonist-mediated responses upon binding to a receptor. The binding of an “antagonist” disrupts the interaction and inhibit the function of an “agonist” at receptors. “Antagonists” mediate their effects by binding to the active site or to allosteric sites on receptors, or they may interact at unique binding sites not normally involved in the biological regulation of the receptor's activity. “Antagonist activity” may be reversible or irreversible. The majority of drug antagonists achieve their potency by competing with endogenous ligands or substrates at structurally defined binding sites on receptors.

A “p75^{NTR} antagonist” is a chemical, agent or ligand that disrupts the interaction with a p75NTR agonist, inhibits the function of p75^{NTR} agonists or inhibits p75NTR mediated signal transduction.

“Response,” e.g., of a cell, tissue, organ, or organism, encompasses a change in biochemical or physiological behaviour, e.g., concentration, density, adhesion, or migration within a biological compartment, rate of gene expression, protein translation, activation or inhibition (e.g. phosphorylation) or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signalling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton.

“Proliferative activity” encompasses an activity that promotes, that is necessary for, or that is specifically associated with, e.g., normal cell division, as well as cancer, tumours, dysplasia, cell transformation, metastasis, and angiogenesis.

“Administration” and “treatment,” as it applies to treatment of a human subject, research subject, veterinary subject, animal, or cell, refers to contact of a pharmaceutical, therapeutic, diagnostic agent or composition, or placebo, to the human subject, animal, or cell. Treatment of a cell encompasses contact of a reagent to the cell, as well as contact of a reagent to a fluid, where the fluid is in contact with the cell.

“Administration” and “treatment” also encompass ex vivo treatment, e.g., ex vivo treatment to a cell, tissue, or organ, followed by contact of the cell, tissue, or organ, to the subject or animal, even where the agent or composition has been metabolized, altered, degraded, or removed, during or after the ex vivo treatment.

“Candidate compound” or “test compound” refers, e.g., to a molecule, complex of molecules, or mixture of molecules, where the candidate compound is used in the development or identification of a therapeutic or diagnostic agent. Testing or screening of a candidate compound is used to determine if the compound can be useful as therapeutic or diagnostic. “Candidate compounds” encompass, e.g., polypeptides, antibodies, natural products, synthetic chemicals, organic compounds, inorganic compounds, nucleic acids and combinations thereof with a second therapeutic or diagnostic, or a carrier, diluent, stabilizer, or excipient.

“Disorder” or “disease” refers to a pathological state, or a condition that is correlated with or predisposes to a pathological state. In particular, “disorder” or “disease” is an impairment of the normal state of the living animal or human body or one of its parts that interrupts or modifies the performance of the vital functions, is typically manifested by distinguishing signs and symptoms, and is a response to environmental factors (as malnutrition, industrial hazards, or climate), to specific infective agents (as worms, bacteria, or viruses), to inherent defects of the organism (as genetic anomalies or impaired functionality of the immune system), or to combinations of these factors.

“Infectious disorder” or “infectious diseases” refers, e.g., to a disorder resulting from a microbe, bacterium, parasite, pathogenic fungus, viruses and the like, as well as to an inappropriate, ineffective, or pathological immune response to the disorder.

“Oncogenic disorder” encompasses a cancer, transformed cell, tumour, dysplasia, angiogenesis, metastasis, and the like, as well as to an inappropriate, ineffective, or pathological immune response to the disorder.

“Effective amount” means, e.g., an amount of a p75^{NTR} agonist, antagonist, or binding compound or composition sufficient to ameliorate a symptom or sign of a disorder, condition, or pathological state.

“Expression” refers to a measure of mRNA or polypeptide encoded by a specific gene. Units of expression may be a measure of, e.g., the number of molecules of mRNA or polypeptide/mg protein in a cell or tissue, or in a cell extract or tissue extract. The units of expression may be relative, e.g., a comparison of signal from control and experimental mammals or a comparison of signals with a reagent that is specific for the mRNA or polypeptide versus a reagent that is non-specific.

“Inflammatory disorder” or “inflammatory disease” means a disorder or pathological condition where the pathology results, in whole or in part, from an increase in the number and/or increase in activation of cells of the immune system, e.g., of T-cells, B-cells, monocytes or macrophages, alveolar macrophages, dendritic cells, NK-cells, NKT-cells, neutrophils, eosinophils, or mast-cells.

An “immune disorder” or “immune disease” is a dysfunction of the immune system. These disorders develop either because the components of the immune system are affected, or because the immune system is overactive or underactive. Furthermore, these disorders can be congenital or acquired.

“Immunotherapy” means the treatment of a disease by inducing, enhancing, or suppressing an immune response. Immunotherapies designed to elicit or amplify an immune response are classified as activation immunotherapies, while immunotherapies that reduce or suppress are classified as suppression immunotherapies.

“Knockout” (KO) refers to the partial or complete reduction of expression of at least a portion of a polypeptide encoded by a gene, e.g., the p75^{NTR} gene, where the gene is endogenous to a single cell, selected cells, or all of the cells of an animal such as a mammal. KO also encompasses embodiments where biological function is reduced, but where expression is not necessarily reduced, e.g., a p75^{NTR} polypeptide comprising an expressed p75^{NTR} polypeptide that contains an inserted inactivating peptide, oligopeptide, or polypeptide. Disruptions in a coding sequence or a regulatory sequence are encompassed by the knockout technique. The cell or mammal may be a “heterozygous knockout”, where one allele of the endogenous gene has been disrupted. Alternatively, the cell or mammal may be a “homozygous knockout” where both alleles of the endogenous gene have been disrupted. “Homozygous knockout” is not intended to limit the disruption of both alleles to identical techniques or to identical outcomes at the genome. Included within the scope of this invention is a mammal in which one or both p75^{NTR} alleles have been knocked out. Suitably, said mammal, in which one or both p75^{NTR} alleles have been knocked out, is a mouse or rat.

“Knock down” (KD) refers to a partial reduction of at least a portion of a polypeptide encoded by a gene, e.g., the p75^{NTR} gene, where the gene is endogenous to a cell line, single cell, selected cells, or all of the cells of an animal such as a mammal. KD is achieved, e.g., by expression of a siRNA/shRNA.

“Transgenic” refers to a genetic change, produced by a technique of genetic engineering that is stably inherited. Transgenic methods, cells, and animals, includes genetic changes that result from use of a knockout technique, a knock-in technique or any other conventional techniques for the production of transgenics.

A “marker” relates to the phenotype of a cell, tissue, organ, animal, e.g., of a mouse, or human subject. A cell surface marker refers to a molecule that is located on the plasma membrane of a specific cell type or even a limited number of cell types. An intracellular marker refers to a molecule that is located inside the cell of specific cell type or even a limited number of cell types.

They are normally used in identification of cell types. Markers are used to detect cells, e.g., during cell purification, quantitation, migration, activation, maturation, or development, and may be used for both in vitro and in vivo studies. An activation marker is a marker that is associated with cell activation.

“Non-human animal” refers to all other animals than a human being. A non-human animal according to the present invention is suitably a mammal or a rodent. More suitably, the non-human animal according to the present invention is selected from a rat, mouse, rabbit, monkey, guinea pig, cat or dog. Most suitably, the non-human animal according to the present invention is a rat or mouse.

“Sensitivity,” e.g., sensitivity of a receptor to a ligand, means that binding of a ligand to the receptor results in a detectable change in the receptor, or in events or molecules specifically associated with the receptor, e.g., conformational change, phosphorylation, nature or quantity of proteins associated with the receptor, or change in genetic or protein expression mediated by or associated with the receptor.

“Soluble receptor” refers to receptors that are water-soluble and occur, e.g., in extracellular fluids, intracellular fluids, or weakly associated with a membrane. Soluble receptor further refers to receptors that are engineered to be water soluble.

“Specificity of binding,” “selectivity of binding,” and the like, refers to a binding interaction between a predetermined ligand and a predetermined receptor that enables one to distinguish between the predetermined ligand and other ligands, or between the predetermined receptor and other receptors. “Specifically” or “selectively” binding, when referring to a ligand/receptor, antibody/antigen, or other binding pair, indicates a binding reaction that is determinative of the presence of the protein in a heterogeneous population of proteins. Thus, under designated conditions, a specified ligand binds to a particular receptor and does not bind in a significant amount to other proteins present in the sample.

A “primary cell” is a cell that is directly derived from the human or animal body.

“CpG oligodeoxynucleotides” (or CpG ODN, short “CpG”) are short single-stranded synthetic DNA molecules that contain a cytosine triphosphate deoxynucleotide followed by a guanine triphosphate deoxynucleotide.

A “gene” encompasses the coding region of a polypeptide and any regulatory sequences, e.g., promoters, operators, enhancers, introns, splice acceptor and donor sites, translational and transcriptional start and stop signals. The coding region may comprise one, continuous exon, or it may comprise more than one exon, i.e., it may be interrupted by one or more introns. A “gene” can encompass one or more open reading frames (ORF).

A “vaccine” is a biological preparation that improves immunity to a particular disease. A vaccine typically contains an ingredient that resembles a disease-causing microorganism and is often made from inactivated forms of the microorganism, its toxins or one of its surface proteins. The ingredient stimulates the body's immune system to recognize the ingredient as foreign, destroy it and memorize it for future infections. Vaccines can be prophylactic (e.g. to prevent or ameliorate the effects of a future infection by a pathogenic microorganism), or therapeutic (e.g., vaccines against cancer).

An “adjuvant” is a pharmacological and/or immunological agent that modifies the effect of other agents. Adjuvants are inorganic or organic chemical entities, macromolecules or entire cells of certain inactivated pathogenic microorganisms, which enhance the immune response to an antigen. They may be included in a vaccine to enhance the immune response to the supplied antigen in a subject, thus minimizing the amount of injected foreign material. Adjuvants can enhance the immune response to the antigen in different ways, e.g., by activation of the Toll-like receptor (TLR) signalling, by extending the presence of an antigen in the blood circulation, by improving the absorption of the antigen by the antigen presenting cells, by activating macrophages and lymphocytes and/or by enhancing the production of cytokines.

### Preferred Embodiments of the Invention

1. Pharmaceutical Composition

The present invention provides a combination of at least one compound selected from an agonist of p75^{NTR} signalling or an antagonist of p75^{NTR} signalling and an activator of a dendritic cell, preferably a PDC.

The invention further provides a pharmaceutical composition comprising said combination of at least one compound selected from an agonist of p75^{NTR} signalling or an antagonist of p75^{NTR} signalling and an activator of a dendritic cell, preferably a PDC and at least one pharmaceutically acceptable carrier or excipient.

The pharmaceutical composition comprising said combination is preferably a vaccine composition.

Said activator of the dendritic cell, preferably the PDC is preferably a TLR receptor agonist, most preferably an agonist selected for TLR7 or TLR9.

The combination of at least one compound selected from an agonist of p75^{NTR} signalling or an antagonist of p75^{NTR} signalling and an activator of a dendritic cell, preferably a PDC, and the pharmaceutical composition comprising said combination are especially suitable for use in immunotherapy, such as the treatment of cancer and infectious diseases. More preferably, said combination or pharmaceutical composition comprising said combination is suitable for use in the treatment of allergic diseases or in allergic desensitization. Even preferably, said combination or pharmaceutical composition comprising said combination is suitable for use in the treatment of autoimmune diseases, chronic inflammatory diseases, GvHD or after transplantation to avoid graft failure.

In a further preferred embodiment antagonists or agonists of p75^{NTR} signalling may be used to induce conditions comprising, but not limited to graft-versus-leukaemia effect (GvL). GvL or graft-versus-tumour effect (GvT) is the beneficial aspect of the graft-versus-host disease. GvL is mainly beneficial in diseases with slow progress, e.g. chronic leukaemia, low-grade lymphoma, and some cases multiple myeloma.

Pharmaceutical compositions suitable for use in this aspect of the invention include compositions wherein the active ingredients are contained in an effective amount to achieve the intended purpose relating to one of the diseases. The determination of a therapeutically effective dose is well within the capability of those skilled in the art and can be estimated initially either in cell culture assays, e. g. of neoplastic cells, or in animal models, usually mice, rats, rabbits, dogs, monkeys or pigs. An animal model may also be used to determine the appropriate concentration range and route of administration. This information is then commonly used to determine useful doses and routes for administration in humans.

A therapeutically effective dose refers to that amount of active ingredient, e.g., an antibody against p75^{NTR}, or an agonist, antagonist or inhibitor of p75^{NTR}, which ameliorates particular symptoms or conditions of the disease. For example, the amount to be administered may be effective to inhibit the activity of the p75^{NTR}. Therapeutic efficacy and toxicity may likewise be determined by standard pharmaceutical procedures in cell cultures or with experimental animals, such as by calculating the ED50 (the dose therapeutically effective in 50% of the population) or LD50 (the dose lethal to 50% of the population) statistics. The dose ratio of toxic to therapeutic effects is the therapeutic index, and it can be expressed as the LD50/ED50 ratio. Pharmaceutical compositions, which exhibit large therapeutic indices, are preferred. The data obtained from cell culture assays and animal studies are used in formulating a range of dosage for human use. The dosage contained in such compositions is preferably within a range of circulating concentrations that include the ED50 with little or no toxicity. The dosage varies within this range depending upon the dosage form employed, the sensitivity of the patient, and the route of administration.

An exact dosage will normally be determined by the medical practitioner in light of factors related to the subject requiring treatment, with dosage and administration being adjusted to provide a sufficient level of the active moiety or to maintain a desired effect. Factors to be taken into account include the severity of the disease state, the general health of the subject, the age, weight, and gender of the subject, diet, time and frequency of administration, drug combination (s), reaction sensitivities, and tolerance/response to therapy. Long-acting pharmaceutical compositions may be administered every 3 to 4 days, every week, or even once every two weeks, depending on the half-life and clearance rate of the particular formulation.

In a preferred embodiment, the present invention provides a method for treating diseases or pathological conditions that are related to p75^{NTR} signalling, preferably of immune diseases, comprising administering a pharmaceutically effective amount of a p75^{NTR} agonist or p75^{NTR} antagonist or of a pharmaceutical composition comprising the same to a subject in need thereof.

Likewise, the invention provides the use of a p75^{NTR} agonist or p75^{NTR} antagonist or of a pharmaceutical composition comprising the same in such methods of treatment.

Moreover, p75^{NTR} agonists or p75^{NTR} antagonists or pharmaceutical compositions comprising the same are provided for use in the treatment of diseases or pathological conditions that are related to p75^{NTR} signalling.

In a further preferred embodiment, the disease or pathological condition that is related to the p75^{NTR} signalling, is selected from the group consisting of central and peripheral neurodegenerative diseases, senile dementia, epilepsy, Alzheimer's disease, Parkinson's disease, Huntington's disease, Down's syndrome, prion diseases, amnesia, schizophrenia, depression, bipolar disorder, amyotrophic lateral sclerosis, multiple sclerosis, cardiovascular conditions, post-ischemic cardiac damage, cardiomyopathies, myocardial infarction, heart failure, cardiac ischemia, cerebral infarction, peripheral neuropathies, damage to the optic nerve and/or to the retina, retinal pigment degeneration, glaucoma, retinal ischemia, macular degeneration, spinal cord traumas, cranial traumas, atherosclerosis, stenosis, wound healing disorders, alopecia, any type of cancer, any type of tumours, any type of metastases, any type of leukemia, respiratory disorders, pulmonary inflammation, allergy, anaphylaxis, asthma, atopic dermatitis, chronic obstructive pulmonary disease, cutaneous pain, somatic pain, visceral pain, neurological pain, chronic neuropathic pain, inflammatory pain, autoimmune diseases, rheumatoid arthritis (polyarthritis, oligoarthritis), ankylosing spondylitis, collagenosis, systemic lupus erythematodes (SLE), SHARP syndrome, Sjögren's syndrome, scleroderma, polymyositis, dermatomyositis, progressive systemic sclerosis, spondyloarthritis (Morbus Bechterew, reactive arthritis, enteropathic arthritis, psoriatic arthritis, undifferentiated spondyloarthritis), rheumatic fever, Aicardi-Goutieres syndrome, vasculitis, Wegener's granulomatosis disease, nephritis, stroke, ulcerative colitis, Crohn's diesease, Morbus Whipple, scleroderma, Still's disease, bronchopulmonary dysplasia (BPD), bronchiolitis, RSV-associated bronchiolitis, Diabetes mellitus, fibromyalgia syndrome, coeliac disease, Hashimoto's disease, hypothyroidism, hyperthyroidism, Addison's disease, graft versus host disease (GVHD), autoimmune thrombocytopenia, autoimmune hemolytic anemia, Löfgren syndrome, Behcet disease, nephrotic syndrome, uveitis, psoriatic arthritis, psoriasis (plaque psoriasis, pustular psoriasis), bone fractures, bone diseases, osteoporosis and all bacterial, fungal, viral infectious diseases, as well infections with eukaryotic parasites.

In a further preferred embodiment, the invention provides a method of monitoring efficacy of the therapy diseases or pathological conditions that are related to p75^{NTR} signalling in a subject comprising the following steps:
measuring T-cell activation such as T-cell cytokine expression, T-cell proliferation, induction of antigen specific T-cell clones, induction of cytotoxic T-cells and/or induction of regulatory T-cells in samples taken on two or more occasions from the subject; and
comparing the level of T-cell cytokines, proliferated T-cells, antigen specific T-cell clones, induction of cytotoxic T-cells and/or regulatory T-cells in a sample taken from the subject with the level present in a sample taken from the subject prior to commencement of a therapy, and/or a sample taken from the subject at an earlier stage of a therapy.

Samples can be taken at intervals over the remaining life, or a part thereof, of a subject. i.e. the biological samples for monitoring the efficacy of a therapy can be taken on two or more occasions. Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-proliferative disease therapy, such as a chemotherapy. In a preferred embodiment, the method of monitoring comprises detecting a change in the amount of T-cell cytokines, proliferated T-cells, antigen specific T-cell clones, induction of cytotoxic T-cells and/or regulatory T-cells in samples taken on two or more occasions.

P75^{NTR} on DCs, most preferably PDCs seems to function as a master switch in the regulation of immune responses. The modulation of immune responses is the major function of vaccine adjuvants. Therefore agonists and antagonists of p75^{NTR} provide a means for novel adjuvants.

Activation of p75^{NTR} on PDCs, most preferably a TLR7 or TLR9 activated PDCs strongly induce Th2 immune responses. Therefore agonists can boost immunization responses in Th2 dependent vaccines. The directed immune response is similar to aluminium salts but works without inducing local inflammation. P75^{NTR} agonists might be used to replace current vaccine adjuvants or could be used in combination to further boost a vaccine response.

In a further embodiment, the present invention thus relates to a vaccine composition comprising a modulator of p75^{NTR} signalling, i.e. an agonist or antagonist of p75^{NTR} signalling. Preferably, p75^{NTR} signalling is modulated in p75^{NTR} expressing dendritic cells, most preferably in p75^{NTR} expressing PDCs.

In a preferred embodiment, the invention provides the use of a vaccine composition comprising a p75^{NTR} agonist for modulating immune responses comprising but not limited to stimulation of Th2 immune responses, suppression of Th1 immune responses, suppression of Th17 immune responses, suppression of regulatory T-cell induced tolerance and the like.

Preferred p75^{NTR} agonists for use in the vaccine composition of the invention are selected from the group comprising NGF, BDNF, NT-3, NT-4, NT-5 and the like.

Further preferred p75^{NTR} agonists, which are suitable for use in the vaccine composition of the invention are selected from activating antibodies, such as anti-p75^{NTR} antibody MC192 (Kimpinski et al., Neurosci 1999, 93:253-263), activating peptides and activating small molecules (e.g. LM11A and derivative compounds, comprising but not limited to LM11A-24 caffeine or LM11A-31 isoleucine, LM11A-36) or are encoded by a nucleic acid.

In a further preferred embodiment, the invention provides the use of a vaccine composition comprising a p75^{NTR} antagonist for modulating immune responses comprising but not limited to suppression of Th2 immune responses, stimulation of Th1 immune responses, stimulation of Th17 immune responses, suppression of regulatory T-cell induced tolerance and the like.

Preferred p75^{NTR} antagonists for use in the vaccine composition of the invention are selected from the group comprising pro-NGF, pro-BDNF, pro-NT-3, pro-NT-4, pro-NT-5 and the like.

Further preferred p75^{NTR} antagonists, which are suitable for use in the vaccine composition of the invention are selected from blocking antibodies (anti human p75^{NTR} monoclonal antibody clones: ME20.4, ME24.1, MLR-1, MLR2, MLR3, HB-8737, NGFR5 and derivatives and humanized versions thereof; anti mouse p75^{NTR} monoclonal antibody: REX, AB 1554; antibodies that prevent binding of neurotrophins to p75^{NTR}: MAb 911, MAb 912 and MAb 938, derivatives and humanized versions thereof, including Tanezumab a humanized version of MAb 911, PG110, REGN475, Fulranumab, MEDI-578), blocking peptides (PEP5, tat-PEP5, C30-35), blocking proteins (protein that prevent binding of neurotrophins to p75^{NTR}: extracellular domain of p75^{NTR}) and small molecule inhibitors (derivatives of 2-oxo-alkyl-1-piperazin-2-one; small molecules that prevent binding of neurotrophins to p75^{NTR}: PD 90780, ALE-0540, Ro 08-2750, Y1036) or are encoded by a nucleic acid, such as shRNA, siRNA or RNAi.

Preferred blocking peptides specifically inhibit the binding of TRAF6 to the intracellular domain of p75^{NTR} (peptides that block the interaction of p75^{NTR} with TRAF6 including peptides binding to the protein motif EGEKLHSDSGISVDS (SEQ ID No. 1) from the intracellular domain of p75^{NTR}, TRAF6 decoy peptides comprising the RPTIPRNPK peptide (SEQ ID No. 2).

The vaccine composition of the invention can further comprise modulators of p75^{NTR} signalling in combination with immune stimulating agents, which are, e.g., selected from monophosphoryl lipid A (MPL) and synthetic derivatives thereof, muramyl dipeptide (MDP) and derivatives thereof, oligodeoxynucleotides (such as CpG, etc.), double-stranded RNA (dsRNA), alternative pathogen-associated molecular patterns (PAMPs, such as E. coli heat labile enterotoxin (LT); flagellin), saponins (Quils, QS-21), small-molecule immune potentiators (SMIPs, e.g., Resiquimod [R848]), cytokines, chemokines and antigens from Mycobacterium tuberculosis.

The vaccine composition of the invention can further comprise modulators of p75^{NTR} signalling in combination with insoluble aluminium compounds, calcium phosphate, liposomes, Virosomes®, ISCOMS®, microparticles (e.g., PLGA), emulsions (e.g., MF59, Montanides), virus-like particles and viral vectors.

The vaccine composition of the present invention may further comprise isolated dendritic cells, preferably isolated PDCs, most preferably isolated p75^{NTR} expressing dendritic cells or PDCs. In a preferred embodiment, the isolated dendritic cells are ex vivo incubated with at least one p75^{NTR} signalling modulator prior to the administration of the vaccine composition to a subject.

In a preferred embodiment of the invention, at least one p75^{NTR} agonist is used to prime said isolated dendritic cells, preferably isolated PDCs, to modulate immune responses comprising but not limited to stimulation of Th2 immune response, suppression of Th1 immune response, suppression of Th 17 immune response and suppression of regulatory T-cell induced tolerance.

In a yet preferred embodiment of the invention, at least one p75^{NTR} antagonist is used to prime said isolated dendritic cells, preferably isolated PDCs, to modulate immune responses comprising but not limited to suppression of Th2 immune response, stimulation of Th1 immune response, stimulation of Th 17 immune response and suppression of regulatory T-cell induced tolerance.

Where the agonist or antagonist is encoded by a nucleic acid such as shRNA or siRNA, said nucleic acid is preferably transfected into the dendritic cell, preferably PDCs, leading to overexpression of the agonist or antagonist in the dendritic cell.

In a further preferred embodiment, vaccine compositions comprising an agonist of p75^{NTR} signalling selected from the group comprising NGF, BDNF, NT-3, NT-4, NT-5 or an antagonist of p75^{NTR} signalling selected from the group comprising pro-NGF, pro-BDNF, pro-NT-3, pro-NT-4, pro-NT-5.

Examples for antagonists of p75^{NTR} signalling, which are suitable for use in the vaccines of the invention and/or for use in therapy, preferably immunotherapy according to the invention are selected from the groups comprising:
Anti human p75^{NTR} Monoclonal antibodies, such as clones ME20.4, ME24.1, MLR-1, MLR2, MLR3, HB-8737, NGFR5, derivatives and humanized versions of the aforementioned antibodies;
Anti murine p75^{NTR} monoclonal antibodies, such as REX, AB1554;
Peptides or peptide derivatives, such as PEP5, tat-PEP5, C30-35, peptides that block the interaction of p75^{NTR} with TRAF6 including peptides binding to the protein motif EGEKLHSDSGISVDS (SEQ ID No. 1) from the intracellular domain of p75^{NTR}, TRAF6 decoy peptides comprising the RPTIPRNPK peptide (SEQ ID No. 2);
Small molecules such as derivatives of 2-oxo-alkyl-1-piperazin-2-one, derivatives of naphthalimide
siRNAs, shRNAs, morpholinos that block expression of p75^{NTR} or downstream signalling;
Nucleic acids coding for peptides or proteins that inhibit p75^{NTR} signalling;
Neurotrophin antagonists that prevent binding of NGF or BDNF to p75^{NTR}, such as:
   Antibodies: MAb 911, MAb 912 and MAb 938, derivatives and humanized versions of the aforesaid antibodies, including Tanezumab (a humanized version of MAb 911), PG110, REGN475, Fulranumab, and MEDI-578;
   Proteins or peptides such as p75^{NTR} extracellular domain;
   Small molecules, such as PD 90780, ALE-0540, Ro 08-2750, and Y1036.

Examples for agonists of TLR7 and/or TLR9, which are suitable for use in the vaccines of the invention and/or for use in therapy, preferably immunotherapy according to the invention are selected from the groups comprising:
Specific Activators of Toll like receptors comprising:
   TLR7 agonists, such as single stranded RNAs, CL075, CL097, CL264, CL307, Gardiquimod, Imiquimod, Loxoribine, Poly(dT) and R848;
   TLR9 agonists, such as:
      CPG-ODNs Class A, such as ODN 1585, ODN 2216, ODN 2336;
      CPG-ODNs Class B, such as ODN BW006, ODN D-SL01 ODN 1668; ODN 1826, ODN 2006, ODN 2007;
      CPG-ODNs Class C, such as ODN D-SL03, ODN 2395, ODN M362;
Live or attenuated viruses, bacteria, parasites;
Viral, bacterial or parasitic extracts.

Examples for agonists of p75^{NTR} signalling, which are suitable for use in the vaccines of the invention and/or for use in therapy, preferably immunotherapy according to the invention are selected from the groups comprising:
Neurotrophins, such as NGF, NGF-Delta 9/13 mutant, BDNF, NT-3, NT-4, NT-5, proNGF, proBDNF, proNT-3, proNT-4, pro-NT-5;
Neurotrophin derived peptides, peptidomimetics, peptoids;
Small molecules such as LM11A and derivative compounds, comprising but not limited to LM11A-24 caffeine or LM11A-31 isoleucine;
Nucleic acids coding for p75^{NTR}, constitutively active p75^{NTR}, or fragments thereof.

2. Cell Based Assay

The present invention provides a cell based assay comprising a non-human animal, or a human or animal primary cells or cell lines that express the nerve growth factor receptor p75^{NTR}, characterized in that the effect of agonism or antagonism of p75^{NTR} signalling on said cell or cell line is measured.

In a preferred embodiment, the present invention provides a cell based assay comprising a non-human animal, or a human or animal primary cells or cell lines that express the nerve growth factor receptor p75^{NTR} and/or at least one protein selected from the group consisting of TLR9, TLR7, TRAF3 and TRAF6 and signalling molecules of the Toll-like receptor pathway, comprising but not limited to MyD88, IRAK1 to 4, IRF3, IRF7, wherein the effect of agonism or antagonism of p75^{NTR} signalling on said cell or cell line is measured.

Suitably, the primary cells used in the assay of the invention are PDCs. Further suitably, the cell lines used in the assay of the invention are derived from PDCs or resemble PDC characteristics.

In a preferred embodiment, there are non-human animals, transgenic cells or cell lines in the assay of the invention, which have been genetically modified to overexpress either p75^{NTR} and/or Toll like receptors TLR7 and/or TLR9, or modified to elicit reduced expression thereof using siRNA, shRNA, morpholinos or modified genomic DNA.

PDCs represent an own cell population and have specific functions. PDCs can clearly and unmistakably be distinguished from conventional dendritic cells and dendritic cells differentiated from monocytes or GM-CSF treated bone marrow by different cell surface markers and the receptors of the TLR family. Human PDCs express BDCA-2, BDCA-4, CD45RA and CD123. Murine PDCs express m-PDCA-1, CD45RA, Ly-6C and Siglec-H. Moreover, the PDCs of both species express toll-like receptors TLR7 and TLR9. In contrast, conventional human dendritic cells (CDCs) express BDCA-1 and BDCA-3, but not BDCA-2 and BDCA-4. Moreover, CDCs express TLR 2 and TLR4, but not TLR7 and TLR9. Dendritic cells differentiated from monocytes (moDCs) show an expression of CD16, CD11c, CD11b and CD209 (murine), which is distinguishable from PDCs. Furthermore, moDCs express TLR3 and TLR4, but not TLR7 and TLR9.

It has been shown in animal models for various inflammatory diseases that the expression and activity of p75^{NTR} in PDCs is causative for the diseases phenotype, whereas other types of dendritic cells, such as CDCs and moDCs are not involved in the disease phenotypes.

Moreover, the reactions caused by ligands of p75^{NTR} are different in PDCs compared to CDCs and moDCs. CDCs and moDCs induce the activation and polarization of T-cells of type Th1, whereas PDCs induce the activation and polarization of T-cells of type Th2. The latter is first described herein.

The invention further provides the use of the cell based assay in screening methods for substances that exert agonistic or antagonistic effects on p75^{NTR} signalling.

In a further embodiment, the invention provides a screening method for agonists and antagonists of the p75^{NTR} signalling.

In a preferred embodiment, the invention provides a screening method for agonists and antagonists of p75^{NTR} signalling comprising the steps of:
Contacting a human or animal primary cell, or cell line that expresses the nerve growth factor receptor p75^{NTR}, with a test substance under;
Incubating said contacted human or animal primary cell or cell line for a period of time, which is sufficient for effecting p75^{NTR} signalling;
Determining the effect of the test substance on the primary cell or cell line;
Comparing the effect of the test substance in the contacted primary cell or cell line with the effect in control cells; and
Selecting a test substance that agonizes or antagonizes p75^{NTR} signalling.

Suitably, the control cells or cell lines are primary cells, most suitably PDCs.

The step of contacting a human or animal primary cell or cell line that expresses the nerve growth factor receptor p75^{NTR}, with a test substance, is preferably performed under conditions allowing the interaction of the test substance with the p75^{NTR} protein. Further preferably, the step of contacting a human or animal primary cell or cell line that expresses the nerve growth factor receptor p75^{NTR}, with a test substance, may be performed under conditions allowing the interaction of the test substance with the p75^{NTR} protein and/or the interaction of the test substance with upstream or downstream factors in the p75^{NTR} signalling pathway.

Control cells are preferably cells or cell lines that have not been contacted with the test agent.

More preferably, control cells are cells or cell lines that do not express p75^{NTR} or that express p75^{NTR} in a reduced amount. Said control cells or cell lines that do not express p75^{NTR} or that express p75^{NTR} in a reduced amount may optionally be contacted with the test substance.

In a further embodiment, the primary cells or cell lines are pre-activated prior to or during their use in the assay and screening methods of the invention. Suitable for use in the pre-activation of the primary cells and cell lines are agonists of TLR7 or TLR9 signalling. Preferred agonists of TLR7 or TLR9 signalling are for example single stranded RNA, CPG oligodeoxynucleotides, Imiquimod, Resiquimod, Gardiquimod, nucleoside analogues, viral or bacterial preparations and the like. Further suitable examples of agonists of TLR7 comprise TLR7 agonists: Single stranded RNAs, CL075, CL097, CL264, CL307, Gardiquimod, Imiquimod, Loxoribine, Poly(dT) and R848. Further suitable examples of agonists of TLR9 comprise TLR9 agonists CpG-ODNs Class A, such as ODN 1585, ODN 2216, ODN 2336; CpG-ODNs Class B such as ODN BW006, ODN D-SL01 ODN 1668, ODN 1826, ODN 2006, ODN 2007 and CpG-ODNs Class C, such as ODN D-SL03, ODN 2395, ODN M362.

The test substance used in the assay and/or screening methods of the invention may be a natural p75^{NTR} agonist, such as nerve growth factor (NGF), Brain-derived neurotrophic factor (BDNF), neurothrophin-3 (NT-3), neurothrophin-4 (NT-4) or neurotrophin-5 (NT-5) or a combination thereof.

The test substance used in the assay and/or screening methods of the invention may also be a precursor of a natural p75^{NTR} agonist, such as pro-NGF, pro-BDNF, pro-NT-3, pro-NT-4, pro-NT5 or a combination thereof.

The method may be performed in presence or absence of a natural ligand of p75^{NTR}, such as a p75^{NTR} agonist or a p75^{NTR} antagonist. If the method is performed in presence of a natural ligand of p75^{N}R, the method is suitably performed under conditions allowing the interaction of the substance with the p75^{NTR} protein or the interaction of the test substance with said natural ligand of p75^{NTR}.

The antagonistic or agonistic effect of the test substance on the p75^{NTR} signalling in the assay and/or screening methods of the invention can be measured based on the expression analysis of cytokines. Suitable cytokines for expression analysis in the assay and/or screening methods of the invention comprise for example interferon alpha (IFNα), tumour necrosis factor alpha (TNFα), interleukin-4 (IL-4), interleukin-5 (IL-5), interleukin-6 (IL-6), interleukin-13 (IL-13) and other cytokines. Similar to the observed effects of NGF an agonist is expected to inhibit the expression of Th1 cytokine IFNα, while expression of Th2 cytokines IL-4, IL-5, IL-6, IL-13 and TNFα is increased. Antagonists would be expected to inhibit described agonistic effects of neurotrophins, where the neurotrophin can be derived from autocrine production by the test cell or externally supplemented. An antagonist is expected to shift the cytokine production to a Th1 profile with strong induction of IFNα and a suppressed or reduced expression of inflammatory Th2 cytokines, e.g. IL-4, IL-5, IL-6, IL-13 and TNFα.

The antagonistic or agonistic effect of the test substance on the p75^{NTR} signalling in the assay and/or screening methods of the invention can be further measured based on analyzing intracellular signalling cascade, for instance their proteins for expression level and their activation, e.g. phosphorylation. Suitable intracellular signalling cascades for analysis in the assay and/or screening methods of the invention comprise for example but not limited to the activation of TNF receptor associated factors (TRAF) 3 and 6, the activation of NF-kappa-B essential modulator (NEMO), the activation of IκB kinase (IKK), the activation of interferon regulatory factor (IRF) 3 and 7, the activation of NF-κB (nuclear factor ‘kappa-light-chain-enhancer’ of activated B-cells) and the like. Similar to observed effects of NGF an agonist is expected to inhibit the activation of IRF3 and IRF7, while IKK and NF kappa (canonical and non-canonical pathways) are activated. An antagonist would inhibit described agonistic effects of neurotrophin where the neurotrophin can be derived from autocrine production by the test cell or externally supplemented.

The antagonistic or agonistic effect of the test substance on the p75^{NTR} signalling in the assay and/or screening methods of the invention can be further determined based on surface marker and intracellular marker expression. Suitable surface marker for expression analysis of either human or murine cells in the assay and/or screening methods of the invention comprise for example Major Histocompatibility Complex proteins of Class I (MHC-I) and/or of Class II (MHC-II), CD80, CD83, CD86, Blood dendritic cell antigen (BDCA) 2 and 4, interleukin-3 receptor alpha (CD123), TLR7, TLR9 and the like. Based on observed effects with NGF an agonist is expected to inhibit upregulation of MHC molecules on the cell surface in environments that favour Th1 reactions, in environments that favour Th2 reaction agonists are expected to increase surface expression of MHC molecules. An antagonist would inhibit described agonistic effects of neurotrophin, where the neurotrophin can be derived from autocrine production by the test cell or externally supplemented

The antagonistic or agonistic effect of the test substance on the p75^{NTR} signalling in the assay and/or screening methods of the invention can be further determined based on measuring uptake, intracellular processing and presentation of external antigens. Suitable external antigens for analysis in the assay and/or screening methods of the invention comprise for example CpG oligodeoxynucleotides, Imiquod, ovalbumin, virus preparations, bacterial preparations, artificial peptide or protein purifications and the like. An agonist leads to increased uptake of external antigens by PDCs and an increased antigen epitope presentation on MHC-I and -II molecules to effector cells, resulting in an intensified effector cell response. An antagonist leads to reduced antigen uptake and presentation, therefore limiting effector cell response.

3. Co-Incubation with T-Cells

The primary cells or cell lines that express p75^{NTR} and which are used in the assay and/or screening methods of the invention, may also be co-incubated with other cells that play a central role in cell-mediated immune responses. Preferred for use in said co-incubation are T-cells.

In a preferred embodiment, the invention provides a screening method for agonists and antagonists of the p75^{NTR} signalling comprising the steps of:
Contacting a human or animal primary cell or cell line that expresses the nerve growth factor receptor p75^{NTR}, which are co-incubated with T-cells, with a test substance;
Incubating said contacted co-culture of said human or animal primary cell or cell line and said T-cells for a period of time, which is sufficient for effecting p75^{NTR} signalling;
Determining the effect of the test substance on the primary cell or cell line and/or on the T-cells;
Comparing of the effect of the test substance in the contacted primary cell or cell line and/or T-cells with control cells; and
Selecting a test substance that agonizes or antagonizes p75^{NTR} signalling.

The method may be performed in presence or absence of a natural ligand of p75^{NTR}, such as a p75^{NTR} agonist or a p75^{NTR} antagonist. If the method is performed in presence of a natural ligand of p75^{NTR}, the method is suitably performed under conditions allowing the interaction of the substance with the p75^{NTR} protein or the interaction of the test substance with said natural ligand of p75^{NTR}.

The step of contacting a human or animal primary cell or cell line that expresses the nerve growth factor receptor p75^{NTR}, with a test substance, is preferably performed under conditions allowing the interaction of the test substance with the p75^{NTR} protein. Further preferably, the step of contacting a human or animal primary cell or cell line that expresses the nerve growth factor receptor p75^{NTR}, with a test substance, may be performed under conditions allowing the interaction of the test substance with the p75^{NTR} protein and/or the interaction of the test substance with upstream or downstream factors in the p75^{NTR} signalling pathway.

Control cells are preferably cells or cell lines that have not been contacted with the test agent.

In a preferred embodiment, control cells are used which do not naturally express p75^{NTR}. This allows that the effect of the test substance on the p75^{NTR} signalling can be directly and unambiguously attributed to the target p75^{NTR}. Moreover, possible side effects of the test substance on other targets can be recognized. Accordingly, test agents that show an agonistic or antagonistic effect on the p75^{NTR} signalling with high specificity and without unwanted side effects can be screened and selected for further development.

In a further embodiment, PDCs in which p75^{NTR} is knocked out or knocked down are used as control cells. Likewise, the level of p75^{NTR} in the control cells can be reduced otherwise. Further suitable according to the invention is the use of PDCs, in which the expression of p75^{NTR} is reduced or inhibited, as control cells.

Said control cells or cell lines that do not naturally express p75^{NTR} or in which p75^{NTR} is knocked out or knocked down may optionally be contacted with the test substance.

The antagonistic or agonistic effect of the test substance on the p75^{NTR} signalling in the assay and/or screening methods of the invention can, alone or in addition to the aforementioned parameters, be further determined based on the stimulation of the co-incubated T-cells. T-cell activation can suitably be detected by determining T-cell cytokine expression such as for example chemokines, interferons, interleukins, lymphokines and tumour necrosis factor; T-cell proliferation; induction of antigen specific T-cell clones and/or induction of regulatory T-cells.

T-cell proliferation can be measured as described herein in example 4.

Induction of antigen specific T-cell clones can be measured by their specific cytokine secretion or their proliferation. Upon contact to antigens i.e. in co-cultures with antigen presenting cells (i.e. DCs) T-cell clones secrete a pattern of cytokines. Specific cytokines for a Th1 response are IFNγ and IL-2, for Th2 IL-4, IL-5 and IL-13, for Th17 IL-17, IL-21 and IL-22 and for regulatory T-cells IL-9, IL-10 and TGFβ. T-cell cytokine secretion can be measured with ELISA, cytometric bead arrays (CBA) or ELISPOT analysis. Proliferation of T-cells can be measured by intensity quantification of fluorescent dye incorporated by T-cells (i.e. CFSE) and his intensity loss during T-cell proliferation using flow cytometry.

Induction of regulatory T-cells can be determined by co-culture assays with PDCs (Gehrie et al., Methods Mol Biol, 2011). In brief, naïve T-cells were isolated from mouse spleen or human peripheral blood via magnetic bead separation (CD4+CD25−). T-cells were co-cultured with PDCs in the presence of anti-CD3 mAb (150 ng/mL), 10 ng/mL IL-2, and 10 ng/mL TGFβ. After 96 hours T-cells were stained with antibodies against CD4, CD25 and FoxP3 (intracellular) to determine the number of activated regulatory T-cells. In parallel, cytokine secretion (i.e. IL-10) of regulatory T-cells can be measured in the supernatant by ELISA.

To investigate the antagonistic or agonistic effect of the test substance on the p75^{NTR} signalling in vivo, the primary cells or cell lines, which express p75^{NTR} and/or at least one of TLR7 and/or TLR9 may be administered to animal models. The test substance may be administered to the same animals prior to, together with or after administration of the primary cells or cell lines.

Moreover, the primary cells or cell lines may also be pre-incubated with the test substance in vitro prior to administration to the animal models.

4. Animal Models

In a further embodiment of the present invention, the primary cells or cell lines, which express p75^{NTR} and/or at least one of TLR7 and/or TLR9, may be used in vivo, i.e. administered to animal models, which are specific for immune, inflammatory or proliferative diseases. Suitable animal models are selected from, for example, from OVA induced allergic asthma models, other models of allergic diseases, EAE models in mouse or rat, diabetes models, SLE models, transplantation models, GvHD models, tumour models and the like.

Suitable allergic asthma models are for example BALB/c and C57BL/6 mice. BALB/c mice typically mount Th2-dominated immune responses, and the induction of parameters of allergic responses such as allergen-specific IgE, airway hyperresponsiveness (AHR), and eosinophilic airway inflammation are robust. Conversely, C57BL/6 mice exhibit Th1-dominated immune responses, and have limitations in the development of allergic airway responses compared with BALB/c mice especially in the development of allergen-specific IgE responses and airway responsiveness to inhaled methacholine. Surprisingly, in response to allergen challenge, for example to ovalbumin (OVA), they do develop a robust BAL eosinophilic response, and in the tissue tend to accumulate more eosinophils in the parenchyma than around the airways, in contrast to BALB/c mice where eosinophils accumulate around the airways.

Experimental autoimmune encephalomyelitis (EAE) is the most commonly used experimental model for the human inflammatory demyelinating disease, multiple sclerosis (MS). EAE is a complex condition in which the interaction between a variety of immunopathological and neuropathological mechanisms leads to an approximation of the key pathological features of MS: inflammation, demyelination, axonal loss and gliosis. The counter-regulatory mechanisms of resolution of inflammation and remyelination also occur in EAE, which, therefore can also serve as a model for these processes. Well known in vivo EAE models are for example the C57BL/6 mouse, where immunization with MOG35-55 in complete Freund adjuvant (CFA) can induce monophasic or a chronic, sustained form of EAE. The former is characterized by multifocal, confluent areas of mononuclear inflammatory infiltration and demyelination in the peripheral white matter of the spinal cord. Macrophages and CD4+ T-cells are the main cell types in the inflammatory infiltrate. Other EAE models are SJL/J mice (immunization with PLP139-151), the Lewis rat (active and passive EAE induced by myelin basic protein (MBP) or transfer of MBP-specific T-cells), and the Dark Agouti (DA) rat (syngeneic spinal cord tissue or recombinant rat MOG can be used to induce EAE).

Of particular interest for the present invention are animal models of immune mediated diabetes (Type 1A). Spontaneous type 1 diabetes-susceptible models include the non-obese diabetic (NOD) mouse, the BioBreeding Diabetes-Prone (BB-DP) rat, the Komeda Diabetes-Prone (KDP) sub-line of the Long-Evans Tokushima Lean rat Lew.1.WR1 and the Lew.1AR1/Ztm rat. Multiple experimentally-induced models of type 1 diabetes are available including: 1) T-cell receptor (TCR) transgenic (Tg) and retrogenic mice with the T-cell receptors of naturally occurring diabetogenic clones 2) Neo-antigen (Ag) expression under the control of the rat insulin promoter (RIP) to establish neo-self antigen pancreatic expression that can be the target of autoimmunity, and 3) RIP-driven expression of costimulatory molecules on beta cells. Mice with knockouts of putative islet autoantigens have allowed direct testing of the pathogenic significance of specific target molecules. Strains of mice with mutations of genes associated with type 1 diabetes in man (FoxP3 and AIRE) are being studied (including an autosomal dominant “human” AIRE mutation).

Systemic lupus erythematosus (SLE) is an autoimmune disease that affects multiple organ systems. SLE is characterized by the loss of B and T-cell tolerance to one or more self-antigens, resulting in polysystemic inflammation. The most commonly used mouse strains that develop spontaneous disease include the F1 cross between New Zealand Black and New Zealand White (NZB/W) mice, MRL/lpr mice, and BXSB/Yaa mice. The common immunological and clinical manifestations of SLE in these 3 strains include hyperactive B and T-cells (their presence and interactions with each other are required for disease), high titres of several autoantibodies directed against nuclear antigens, defective clearance of immune complexes, and fatal immune glomerulonephritis.

The limiting factor for successful hematopoietic stem cell transplantation (HSCT) is graft-versus-host disease (GvHD), a post-transplant disorder that results from immune-mediated attack at the recipient tissue by donor T-cells contained in the transplant. The sequence of events that lead to the development of GvHD has largely been defined using mouse models. Early work established that T-cell alloreactivity is the underlying cause of the disease (Korngold and Sprent, 1978; Sprent et al., 1986). The pathology of both acute and chronic mouse models of GvHD relies on T-cell alloreactivity, but each form has a different phenotype owing to differential involvement of cytotoxic (CD8+) or helper (CD4+) T-cell subsets. Donor CD8+ T-cells are activated when their T-cell receptor (TCR) binds to recipient peptides presented in the context of recipient class I major histocompatibility complex (MHC) molecules. Suitable in vivo mouse models are reviewed in Schroeder M. A. & DiPersio J. F., Mouse models of graft-versus-host disease: advances and limitations; Dis Model Mech. 2011 May; 4(3):318-33.

Suitable tumour models are skin cancer model (B16 melanoma) or fibrosarcoma cancer model (cell line MCA-102 or MCA-207). After injection of cancer cell lines or primary tumour cells C57BL/6 or BALB/c mice develop cancers. T-cells and DC, preferably PDCs, are incubated with tumour cell lysate in vitro. Afterwards T-cells alone or in combination with DCs are injected into the cancer cell bearing mouse. Efficiency of PDC immunization is measured via quantification of metastasis development and the development and activity of cytotoxic T-cells. Other tumour mouse models are, e.g., the B16-F10-induced metastatic lung cancer model (Liu et al., JCI, 2008) or the E.G7 T-cell lymphoma model (Lou et al., J Immunol, 2007). In both models, activated PDCs were injected into tumour-bearing mice (tumour cells were injected before), injected before tumour cells were applied or both in parallel. After several days/weeks tumour size can be measured.

Suitable transplantation models are allogeneic organ transplantations in mouse, e.g. skin and cardiac transplantation, bone marrow transplantation, with co-transplantations of DCs preferably PDCs. For example, recipient B10.BR or BA.B10 mice were irradiated with 2 doses of 5.5 Gy separated by 3 hours on day −2. On day 0, recipient mice were transplanted with combinations of 3 to 5×10³ FACS-sorted HSCs, 5×10⁴ FACS-sorted donor pre-PDCs, and 3×10⁵ or 1×10⁶ MACS-purified T-cells from B6 CD45.1 donors. Mice were weighed twice weekly and examined daily for signs of GVHD as described previously. Moribund animals losing more than 25% of initial body weight, and mice surviving until the end of the experiment, were euthanized and tissues were processed for histopathologic analysis of tumour-tropic sites, including liver, small bowel, and large bowel. Flow cytometric chimaerism analyses were performed on blood leukocytes on days 40 (±1), 60 (±2), and 90 (±5) after transplant (Lu Y et al., Blood. 2012 Jan. 26; 119(4):1075-85). Furthermore BALB/c hearts were transplanted as fully vascularised heterotopic grafts into C57BL/6 mice as described. BALB/c cardiac grafts were transplanted by suturing of donor aorta and donor pulmonary artery end-to-side to the C57BL/6 recipient lower abdominal aorta and inferior vena cava, respectively. Recipient mice received intravenous injections in 0.5 ml PBS at various times. For tolerance, mice were treated with DST (1_107 donor splenocytes intravenously) on day −7 and 250 mg mAb to CD40L on days −7, −4, 0 and +4 (times relative to transplantation). One group received 100 mg mAb to CD40L 30 d after toleration and mice rejected at 37-40 d. Graft function was monitored every other day by abdominal palpation. Tolerating mice were studied at 1, 5 and 10 weeks after transplantation. Mice that had graft survival 10 weeks or more were considered ‘tolerated’ (called ‘10-week tolerated’ here). Untreated control mice received hamster IgG in PBS and rejection, defined as complete cessation of a palpable beat and confirmed by direct visualization at laparotomy, occurred 1 week after transplantation (Qian S et al, Hepatology. 1994 19:916-924.

In a further preferred embodiment, the primary cells or cell lines, which express p75^{NTR} and/or at least one of TLR7 and/or TLR9 are pre-incubated with the test substance or p75^{NTR} antagonists and/or agonists in the presence or absence of natural agonists of p75^{NTR} or precursors thereof. Natural agonists of p75^{NTR} are for example nerve growth factor (NGF), Brain-derived neurotrophic factor (BDNF), neurothrophin-3 (NT-3), neurothrophin-4 (NT-4) and neurotrophin-5 (NT-5) or a combination thereof. Precursors of natural p75^{NTR} agonists are for example pro-NGF, pro-BDNF, pro-NT-3, pro-NT-4, pro-NT-5 or a combination thereof.

The test substance can be administered to the animal models via any suitable route. A typical administration is performed orally or intravenously.

The primary cells or cell lines, which express p75^{NTR} and/or at least one of TLR7 and/or TLR9, are typically injected into the blood stream or into specifically desired organs or tissues of the animal models.

In order to determine the antagonistic or agonistic effect of a test substance in vivo, T-cell activation can be detected by determining T-cell cytokine expression such as for example chemokines, interferons, interleukins, lymphokines and tumour necrosis factor; T-cell proliferation; induction of antigen specific T-cell clones and/or induction of regulatory T-cells in samples obtained from the treated animals. The samples are preferably blood samples or tissue samples.

Preferably, said sample and/or control sample has already been obtained from treated animal and/or the control animal prior to the determination of the effect of the test substance in the animal model.

The in vivo determination of the antagonistic or agonistic effect of a test substance is preferably performed in the presence of control animals. More preferably, animals of the same species and/or strain are used as control animals. Most preferably, animals which comprise at least the PDCs but in which p75^{NTR} is not expressed or expressed at a lower level, are used as control animals. This allows that the in vivo effect of the test substance on the p75^{NTR} signalling can be directly and unambiguously attributed to the target p75^{NTR}. Moreover, possible side effects of the test substance on other targets can be recognized. Accordingly, test agents that show an agonistic or antagonistic effect on the p75^{NTR} signalling with high specificity and without unwanted side effects can be screened and selected for further development.

In one embodiment, animals which comprise at least the PDCs but in which p75^{NTR} are knocked out are used as control animals.

Likewise, the level of p75^{NTR} can be reduced otherwise. Further suitable according to the invention is the use of animals, which comprise at least the PDCs but in which the expression of p75^{NTR} is reduced or inhibited, as control animals.

In order to provide appropriate control animals, in one embodiment of the invention, PDCs are administered to the control animals, which do not naturally express p75^{NTR}, or in which the p75^{NTR} gene is knocked out or in which the expression of the p75^{NTR} gene is reduced or inhibited. In another embodiment, the p75^{NTR} gene may be knocked out or the expression of the p75^{NTR} gene may be reduced or inhibited not only in the administered PDCs but also in the endogenous cells of the control animals.

Reduction or inhibition of p75^{NTR} can be achieved e.g. using shRNA, siRNA, antisense nucleotides and the like.

A small hairpin RNA or short hairpin RNA (shRNA) is a sequence of RNA that makes a tight hairpin turn that can be used to silence target gene expression via RNA interference (RNAi).

Expression of shRNA in cells is typically accomplished by delivery of plasmids or through viral or bacterial vectors. Small interfering RNA (siRNA), sometimes known as short interfering RNA or silencing RNA, is a class of double-stranded RNA molecules of about 20-25 base pairs in length. siRNA interferes with the expression of specific genes with complementary nucleotide sequences. siRNA functions by causing mRNA to be broken down after transcription resulting in no translation.

In a preferred embodiment, the invention provides a method for ex vivo determination of the antagonistic or agonistic effect of a test substance in samples which were obtained from the aforesaid animal models and control animals after the aforesaid animal models have received the PDCs and the test substance.

The invention further relates to antagonists and agonists of p75^{NTR} signalling that have been identified with the assay and/or the screening methods of the present invention. Agonists and antagonists—as identified with the methods disclosed herein may include proteins, nucleic acids, carbohydrates, antibodies, or any other molecules; for example, they may include small molecules and organic compounds that bind to p75^{NTR} by a competitive or non-competitive type mechanism. Preferred are small molecule antagonists and agonists of p75^{NTR}.

The specific agonists or antagonists of p75^{NTR}, and agonists of TLR7 and/or TLR9 as used herein are described in table 2.

TABLE 2
Specific agonists or antagonists of p75^{NTR}, and agonists of TLR7 or TLR9

| Compound | Description | Structure/Sequence |
|---|---|---|
| Imiquimod | 1-Isobutyl-1H- imidazo[4,5-c]chinolin-4- amin | 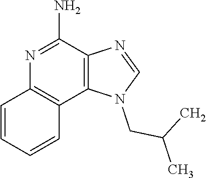 [SMILES: CC(C)Cn1cnc2c(N)nc3ccccc3c21] |
| Resiquimod (R848) | 1-[4-Amino-2- (ethoxymethyl)-1H- imidazo[4,5-c]chinolin-1- yl]-2-methylpropan-2-ol | 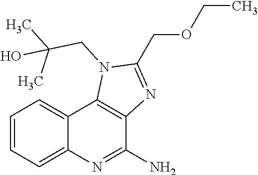 [SMILES: CCOCc1nc2c(N)nc3ccccc3c2n1CC(C)(C)O] |
| Gardiquimod | | 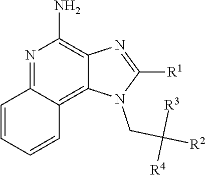 |
| CL264 | 9-benzyl-8 hydroxyadenine derivative | 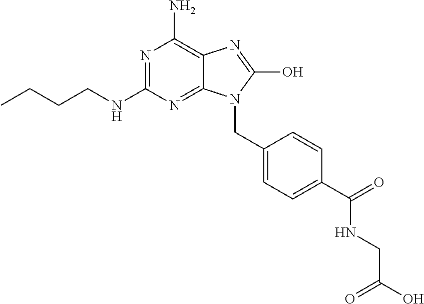 [SMILES: CCCCNc1nc(N)c2nc(O)n(Cc3ccc(C(=O)NCC(=O)O)cc3)c2n1] |
| CL907 | derivative of the imidazoquinoline compound R848 | 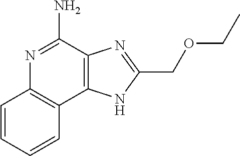 [SMILES: CCOCc1nc2c(N)nc3ccccc3c2[nH]1] |
| CL075 | thiazoloquinolone derivativ | 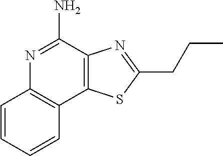 [SMILES: CCCc1nc2c(N)nc3ccccc3c2s1] |
| CL307 | Base analoge; (N1-glycinyl[4-((6- amino-2-(butylamino)-8- hydroxy-9H-purin-9- yl)methyl)benzoyl] spermine) | 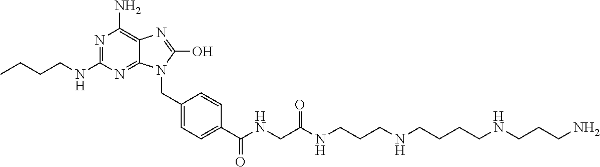 [SMILES: CCCCNc1nc(N)c2nc(O)n(Cc3ccc(C(=O)NCC(=O)NCCCNCCCCNCCCN)cc3)c2n1] |
| Loxoribine | guanosine analog derivatized at position N⁷ and C⁸ | 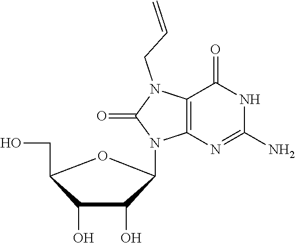 |
| Poly(dT) | a thymidine | |
| | homopolymer | |
| | phosphorothioate ODN | |
| ODN 1585 | Class A synthetic | 5′-ggGGTCAACGTTGAgggggg-3′ (20 mer) |
| | oligonucleotides that | (SEQ ID NO: 5) |
| | contain unmethylated | |
| | CpG dinucleotides in | |
| | particular sequence | |
| | contexts (CpG motifs); | |
| | characterized by a | |
| | phosphodiester central | |
| | CpG-containing | |
| | palindromic motif and a | |
| | phosphorothioate 3′ poly- | |
| | G string | |
| ODN 2216 | Class A synthetic | 5′-ggGGGACGA:TCGTCgggggg-3′ (20 mer) |
| | oligonucleotides that | (SEQ ID NO: 6) |
| | contain unmethylated | |
| | CpG dinucleotides in | |
| | particular sequence | |
| | contexts (CpG motifs); | |
| | characterized by a | |
| | phosphodiester central | |
| | CpG-containing | |
| | palindromic motif and a | |
| | phosphorothioate 3′ poly- | |
| | G string | |
| ODN 2236 | Class A synthetic | 5′-gggGACGAC:GTCGTGgggggg-3′ (21 mer) |
| | oligonucleotides that | (SEQ ID NO: 7) |
| | contain unmethylated | |
| | CpG dinucleotides in | |
| | particular sequence | |
| | contexts (CpG motifs); | |
| | characterized by a | |
| | phosphodiester central | |
| | CpG-containing | |
| | palindromic motif and a | |
| | phosphorothioate 3′ poly- | |
| | G string | |
| ODN BW006 | synthetic oligonucleotide | 5′-tcg acg ttc gtc gtt cgt cgt tc-3′ (23 mer) |
| | that contains | (SEQ ID NO: 8) |
| | unmethylated CpG | |
| | dinucleotides in | |
| | particular sequence | |
| | contexts (CpG motifs); | |
| | type B CpG ODN | |
| | containing twice the | |
| | optimal motif in human, | |
| | GTCGTT[1] | |
| ODN D- | B class double-stem loop | 5′-tcg cga cgt tcg ccc gac gtt cgg ta-3′ (26 mer) |
| SL01 | ODN; is a synthetic | (SEQ ID NO: 9) |
| | oligonucleotide that | |
| | contains unmethylated | |
| | CpG dinucleotides in | |
| | particular sequence | |
| | contexts (CpG motifs) | |
| ODN 1668 | B-class CpG ODN | 5′-tccatgacgttcctgatgct-3′ (20 mer) |
| | specific for mouse TLR9; | (SEQ ID NO: 10) |
| | is a synthetic | |
| | oligonucleotide that | |
| | contains unmethylated | |
| | CpG dinucleotides in | |
| | particular sequence | |
| | contexts (CpG motifs) | |
| ODN 1826 | B-class CpG ODN | 5′-tccatgacgttcctgacgtt-3′ (20 mer) |
| | specific for mouse TLR9; | (SEQ ID NO: 11) |
| | is a synthetic | |
| | oligonucleotide that | |
| | contains unmethylated | |
| | CpG dinucleotides in | |
| | particular sequence | |
| | contexts (CpG motifs) | |
| ODN 2006 | B-class CpG ODN | 5′-tcgtcgttttgtcgttttgtcgtt-3′ (24 mer) |
| | specific for human | (SEQ ID NO: 12) |
| | TLR9; is a synthetic | |
| | oligonucleotide that | |
| | contains unmethylated | |
| | CpG dinucleotides in | |
| | particular sequence | |
| | contexts (CpG motifs) | |
| ODN 2007 | B-class CpG ODN | 5′-tcg tcg ttg tcg ttt tgt cgt t-3′ (22 mer) |
| | specific for | (SEQ ID NO: 13) |
| | bovine/porcine TLR9; is | |
| | a synthetic | |
| | oligonucleotide that | |
| | contains unmethylated | |
| | CpG dinucleotides in | |
| | particular sequence | |
| | contexts (CpG motifs) | |
| ODN D- | C class double-stem loop | 5′-tcg cga acg ttc gcc gcg ttc gaa cgc gg-3′ (29 mer) |
| SL03 | ODN; is a synthetic | (SEQ ID NO: 14) |
| | oligonucleotide that | |
| | contains unmethylated | |
| | CpG dinucleotides in | |
| | particular sequence | |
| | contexts (CpG motifs) | |
| ODN 2395 | C-class CpG ODN | 5′-tcgtcgttttcggcgcgcgccg-3′ (22 mer) |
| | specific for human/mouse | (SEQ ID NO: 15) |
| | TLR9; is a synthetic | |
| | oligonucleotide that | |
| | contains unmethylated | |
| | CpG dinucleotides in | |
| | particular sequence | |
| | contexts (CpG motifs) | |
| ODN M362 | C-class CpG ODN | 5′-tcgtcgtcgttc:gaacgacgttgat-3′ (25 mer) |
| | specific for human/mouse | (SEQ ID NO: 16) |
| | TLR9; is a synthetic | |
| | oligonucleotide that | |
| | contains unmethylated | |
| | CpG dinucleotides in | |
| | particular sequence | |
| | contexts (CpG motifs) | |
| LM11A derivatives | e.g., LM11A-31 (see figure), (2S,3S)-2- Amino-3-methyl-N-[2-(4- morpholinyl)ethyl]pentan amide dihydrochloride | 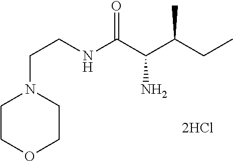 [SMILES: CC[C@H](C)[C@H](N)C(=O)NCCN1CCOCC1] |
| PD90780 | substituted pyrazoloquinazolinone; 7-(Benzoylamino)-4,9- dihydro-4-methyl-9-oxo- pyrazolo[5,1- b]quinazoline-2- carboxylic acid | 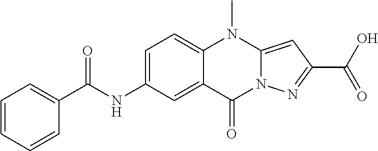 [SMILES: Cn1c2ccc(NC(=O)c3ccccc3)cc2c(=O)n2nc(C(=O)O)cc12] |
| ALE-0540 | 1H- Benz(de)isoquinoline- 1,3(2H)-dione, 2-((2- hydroxyethyl)amino)-5- nitro- | 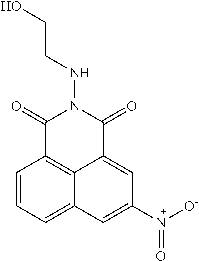 |
| Ro 08-2750 | 2,3,4,10-Tetrahydro- 7,10-dimethyl-2,4- dioxobenzo[g]pteridine- 8-carboxaldehyde | 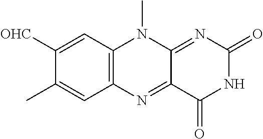 [SMILES: Cc1cc2nc3c(=O)[nH]c(=O)nc-3n(C)c2cc1C=O] |
| YL036 | A furyl- thioxothiazolidinone compound; 3-[4-Oxo-5- [[5-(4-sulfamoylphenyl)- 2-furyl]methylene]-2- thioxo-thiazolidin-3- yl]propanoic acid | 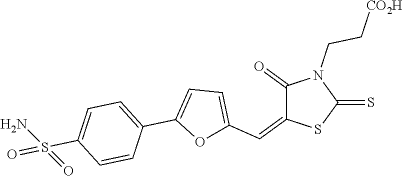 [SMILES: NS(=O)(=O)c1ccc(-c2ccc(/C=C3/SC(=S)N(CCC(=O)O)C3=O)o2)cc1] |
| QS21 | saponin | 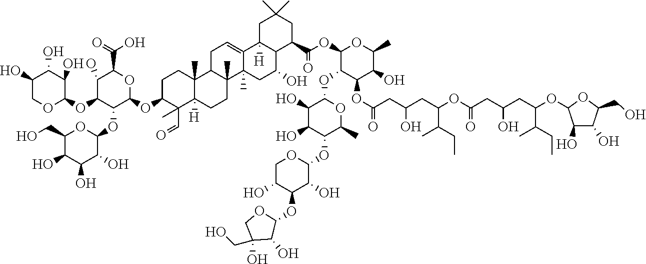 |
| naphtalimide | | 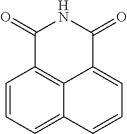 [SMILES: O=C1NC(=O)c2cccc3cccc1c23] |
| derivatives of 2-oxo-alkyl- 1-piperazin- 2-one | | 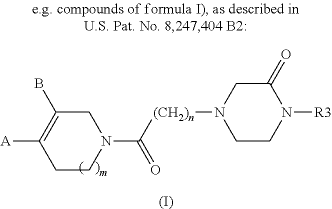 |

A suitable derivative of 2-oxo-alkyl-1-piperazin-2-one is for example a compound selected from the group consisting of:
4-{2-[4-(4-chloro-3-trifluoromethylphenyl)-3,6-dihydro-2H-pyridin-1-yl]-2-oxoethyl}-1-(5-trifluoromethylpyridin-2-yl)piperazin-2-one;
4-{2-[4-(4-chloro-3-trifluoromethylphenyl)-3,6-dihydro-2H-pyridin-1-yl]-2-oxoethyl}-1-(5-methylpyridin-2-yl)piperazin-2-one;
4-{2-[4-(4-chlorophenyl)-3,6-dihydro-2H-pyridin-1-yl]-2-oxoethyl}-1-(5-trifluoromethylpyridin-2-yl)piperazin-2-one;
4-{2-oxo-2-[4-(3-trifluoromethylphenyl)-3,6-dihydro-2H-pyridin-1-yl]ethyl}-1-pyridin-2-ylpiperazin-2-one;
4-{2-[4-(4-chloro-3-trifluoromethylphenyl)-3,6-dihydro-2H-pyridin-1-yl]-2-oxoethyl}-1-pyridin-2-ylpiperazin-2-one;
4-{2-[4-(4-chlorophenyl)-3,6-dihydro-2H-pyridin-1-yl]-2-oxoethyl}-1-pyridin-2-yl-piperazin-2-one;
4-{2-[4-(2,3-dichlorophenyl)-3,6-dihydro-2H-pyridin-1-yl]-2-oxoethyl}-1-(5-trifluoromethylpyridin-2-yl)piperazin-2-one;
4-{2-[4-(4-chlorophenyl)-3,6-dihydro-2H-pyridin-1-yl]-2-oxoethyl}-1-(6-chloropyridin-2-yl)piperazin-2-one;
4-{2-[4-(3-chlorophenyl)-3,6-dihydro-2H-pyridin-1-yl]-2-oxoethyl}-1-(5-trifluoromethylpyridin-2-yl)piperazin-2-one;
4-{2-[4-(4-trifluoromethylphenyl)-3,6-dihydro-2H-pyridin-1-yl]-2-oxoethyl}-1-(5-trifluoromethylpyridin-2-yl)piperazin-2-one;
4-{2-[4-(3-trifluoromethylphenyl)-3,6-dihydro-2H-pyridin-1-yl]-2-oxoethyl}-1-(5-trifluoromethylpyridin-2-yl)piperazin-2-one;
4-{2-[4-(4-chloro-3-trifluoromethylphenyl)-3,6-dihydro-2H-pyridin-1-yl]-2-oxoethyl}-1-pyridin-3-yl-piperazin-2-one;
1-(6-chloropyridin-3-yl)-4-{2-[4-(4-chloro-3-trifluoromethylphenyl)-3,6-dihydro-2H-pyridin-1-yl]-2-oxoethyl}piperazin-2-one;
4-{2-oxo-2-[5-(3-trifluoromethylphenyl)-3,6-dihydro-2H-pyridin-1-yl]ethyl}-1-(5-trifluoromethylpyridin-2-yl)piperazin-2-one;
4-{2-oxo-2-[4-(3-trifluoromethoxylphenyl)-3,6-dihydro-2H-pyridin-1-yl]ethyl}-1-pyridin-2-ylpiperazin-2-one;
4-{2-[4-(4-chloro-3-trifluoromethylphenyl)-2,5-dihydropyrrol-1-yl]-2-oxoethyl}-1-(5-trifluoromethylpyridin-2-yl)piperazin-2-one;
4-{2-[4-(3,5-bistrifluoromethylphenyl)-3,6-dihydro-2H-pyridin-1-yl]-2-oxoethyl}-1-(5-trifluoromethylpyridin-2-yl)piperazin-2-one;
4-{2-[4-(3-methylphenyl)-3,6-dihydro-2H-pyridin-1-yl]-2-oxoethyl}-1-(5-trifluoromethylpyridin-2-yl)piperazin-2-one;
4-{2-[4-phenyl-3,6-dihydro-2H-pyridin-1-yl]-2-oxoethyl}-1-(5-trifluoromethylpyridin-2-yl)piperazin-2-one;
4-{2-oxo-2-[5-(2,3-dichlorophenyl)-3,6-dihydro-2H-pyridin-1-yl]ethyl}-1-(5-trifluoromethylpyridin-2-yl)piperazin-2-one
4-{2-oxo-2-[5-(3-methoxyphenyl)-3,6-dihydro-2H-pyridin-1-yl] ethyl}-1-(5-trifluoromethylpyridin-2-yl)piperazin-2-one;
   suitably in the form of a base or of an acid addition salt.

These compounds and the synthesis thereof are disclosed in US2011144122 A1 and U.S. Pat. No. 8,247,404 B2.

The invention is now further described in the following working examples.

### EXAMPLES OF THE INVENTION

### Example 1: The Neurotrophin NGF Strongly Enhances PDC-Mediated Allergic Asthma in Mice in a p75^{NTR} Dependent Manner

Methods

Mouse Strains

Heterozygous p75^{NTR} knockout mice (p75^{NTR+/-}) were purchased from The Jackson Laboratory (Bar Harbor, Me., USA) and bred under pathogen-free conditions in the animal facility of the TU Dresden. Male and female p75^{NTR+/+} and p75^{NTR-/-} mice were used at 10-12 weeks of age for experiments.

Generation of PDCs

Murine PDC were generated in vitro as follows: Bone marrow cells were isolated by flushing femur and tibia of mice. Erythrocytes were lysed using ACK buffer. Remaining cells were washed and cultured at a density of 2×10⁶ cells per ml in RPMI 1640 medium supplemented with 10% FCS, 1 mM sodium pyruvat, 2 mM L-glutamine, 100 IU per ml penicillin, 100 μg/ml streptomycin, 10 mM HEPES buffer and 0.1 mM β-mercaptoethanol. To differentiate bone marrow cells into PDCs, 100 ng/ml Flt3-L (fms-like tyrosine kinase 3-ligand) was added to the cells. After 8 days of culture PDCs were enriched by removing the CD11b⁺ fraction using CD11b Microbeads (Miltenyi Biotec) according to manufacturer instructions. Dead cells were excluded using Dead cell removal Kit (Miltenyi Biotec). Purity of PDCs was evaluated by flow cytometry (CD11b⁻CD11c⁺B220⁺Siglec-H⁺).

Allergic Asthma Induction in Mice

To induce allergic asthma, in vitro generated PDCs were incubated with 100 μg/ml ovalbumin (OVA; grade V; Sigma-Aldrich) for 24 h. To sensitize mice, 1×10⁶ OVA-loaded PDCs were injected intratracheally to the lungs of anesthetized mice using 24GA i.v. cannula (BD Neoflon™). Control animals received either the same amount of PDCs without OVA or PBS. After 10 days, mice were exposed to 1% w/v OVA-aerosol for 30 min on 3 consecutive days in order to induce allergic reaction. 24 h after last provocation, animals were sacrificed and the immune reaction was examined based on the bronchoalveolar lavage fluid (BALF) cell composition (eosinophils, lymphocytes, macrophages) and pro-inflammatory cytokines spectrum, lung histology and blood serum OVA-specific IgE levels.

BALF Cells Analysis

BALF cells were quantified by flow cytometry. Cells were preincubated with FcR blocking reagent to avoid a non-specific binding. The following antibodies were used for staining: CD3-V500, CD4-V450, CD8-PE Cy7, CD11c-APC Cy7, B220-PE, F4/80-PerCP, SiglecF-AF647, Ly6G-FITC. Among lymphocytes (FSC^{low}/SSC^{low}) the CD4⁺ T helper cells were designated as CD3^{pos}CD4^{pos}; the CD8⁺ T helper cells as CD3^{pos}CD8^{pos}; B-cells as CD3^{neg}B220^{pos}; among granulocytes (FSC^{low}/SSC^{high}, Ly6G^{pos}) eosinophils were assigned as SiglecF^{pos}CD11c^{neg} and neutrophils as SiglecF^{neg}. Macrophages were assigned as FSC^{high} highly autofluorescent CD11c^{pos}F4/80^{pos} cells. Additionally, the cytospins of collected cells were prepared and stained according to Pappenheim's method.

Quantification of BALF Cytokines

BALF supernatant was used for quantification of IL-4, IL-5 and IL-13 by ELISA (eBioscience) according to manufacturer instruction.

Histological Analysis

Lungs were perfused with PBS and fixed in 4% v/v formaldehyde. 4 μm sections of paraffin-embedded lungs were stained with PAS staining for quantification of inflammation and GobleT-cell hyperplasia.

### Example

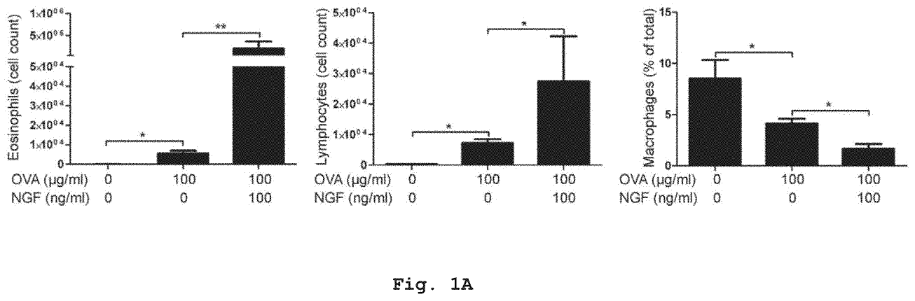

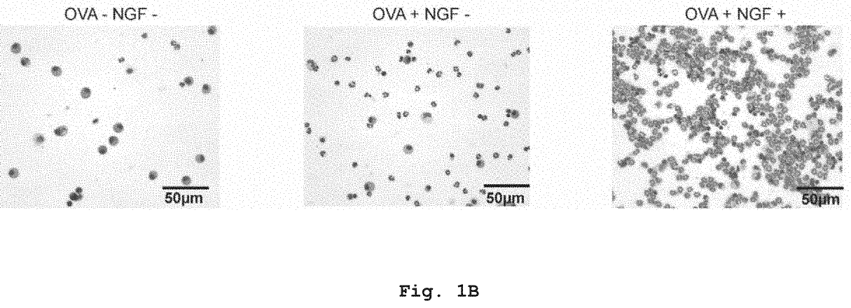

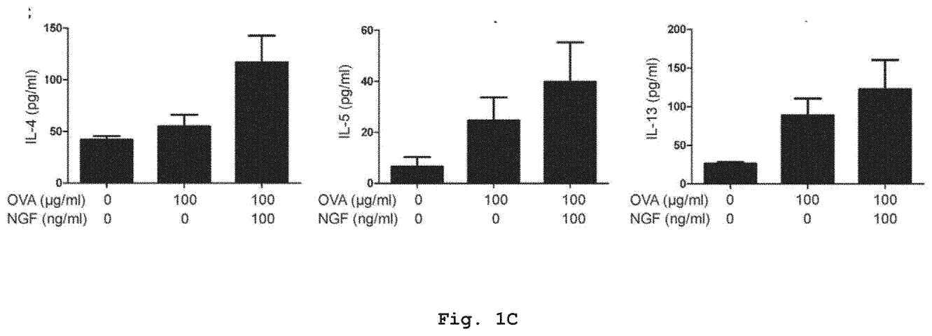

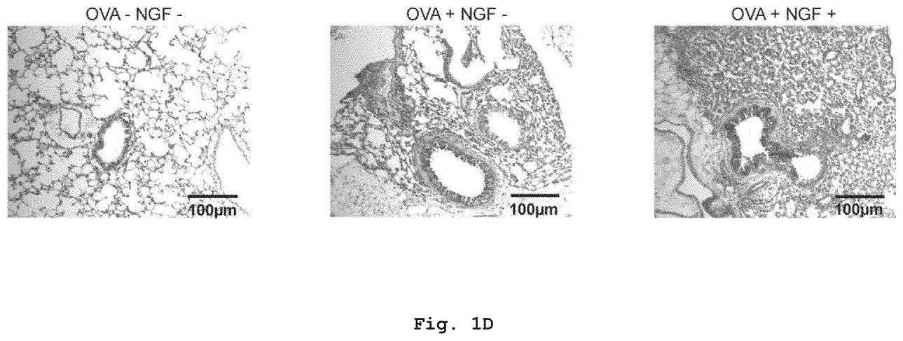

NGF is present in the lung and increased during inflammatory processes such as allergic asthma. To investigate the impact of NGF on PDCs during allergen-mediated immune response, p75^{NTR+/+} PDCs were incubated with ovalbumin (OVA) in the presence or absence of NGF (100 ng/ml) prior intratracheal instillation to p75^{NTR+/+} mice. In the BALF, numbers of eosinophils and lymphocytes were significantly augmented when the OVA up-take by PDCs was carried out in the presence of NGF compared to PDCs incubated with OVA alone (FIGS. 1A, 1B). Furthermore, OVA-loaded PDCs treated with NGF caused increased production of Th2 cytokines (IL-4, IL-5 and IL-13) in the lung in comparison to PDCs pulsed with OVA in the absence of NGF (FIG. 1C). Histological lung sections from mice that received OVA-loaded PDCs showed increased perivascular inflammation and enhanced mucus production (FIG. 1D). Treatment of PDCs with NGF during OVA-uptake potentiated the inflammatory phenotype in the lung (FIG. 1D). In contrast, p75^{NTR-/-} PDCs loaded with OVA in the presence or absence of NGF were not able to induce airway inflammation (data not shown). Our data indicate that NGF triggers p75^{NTR}-expressing PDCs into a pro-inflammatory phenotype, leading to much severe airway inflammation in the asthma model.

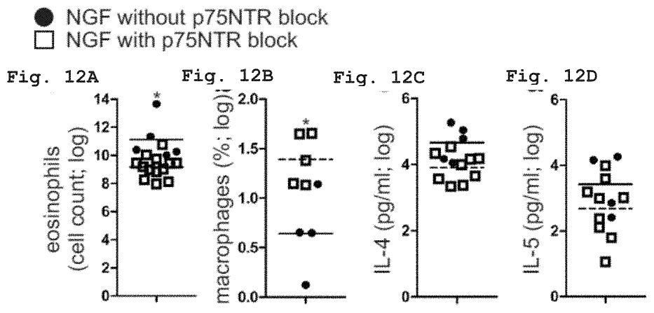

To substantiate the p75^{NTR} dependent impact of NGF on PDCs during allergen-mediated immune response, p75^{NTR+/+} PDCs were incubated with ovalbumin (OVA) and NGF (100 ng/ml) in the presence or absence of the p75^{NTR}-specific inhibitory peptide PEP5 prior intratracheal instillation to p75^{NTR+/+} mice. In the BALF, numbers of eosinophils and macrophages were significantly reduced when the OVA up-take by NGF-stimulated PDCs was carried out in the presence of PEP5 compared to PDCs incubated without PEP5 (FIGS. 12A, 12B). Furthermore, OVA-loaded and NGF-stimulated PDCs treated with PEP5 caused reduced production of Th2 cytokines (IL-4, IL-5, IL-13) in the lung in comparison to PDCs pulsed in the absence of PEP5 (FIGS. 12C, 12D).

### Example 2: p75^{NTR} Knockout Inhibits Th2 Immune Responses and Blocks Tolerance Development

Methods

as described above

### Example

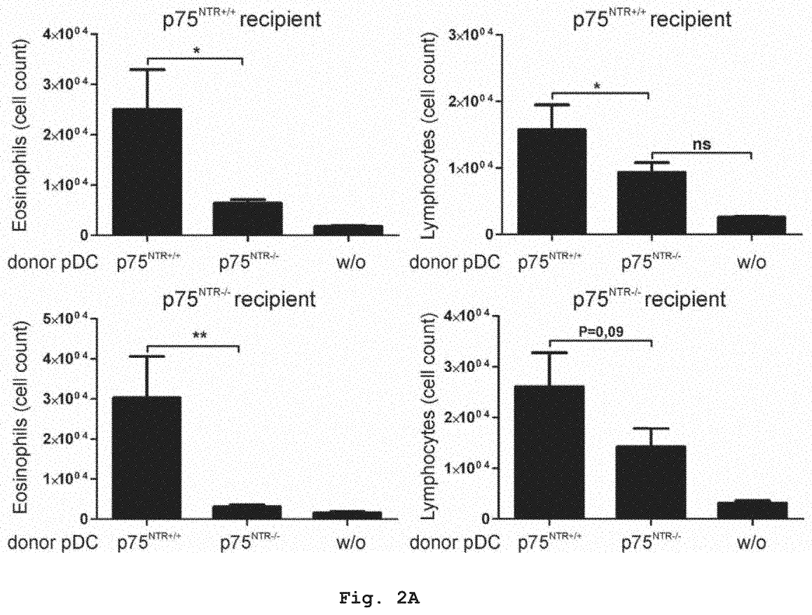

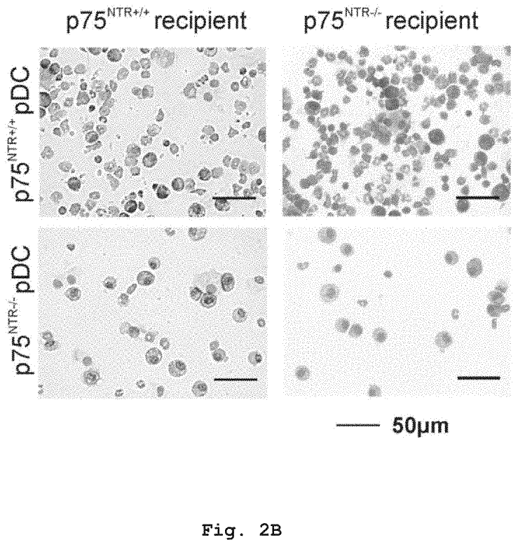

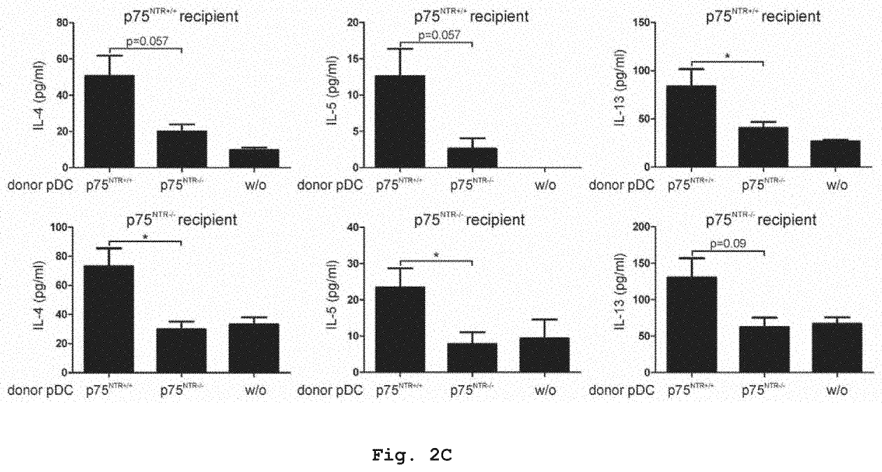

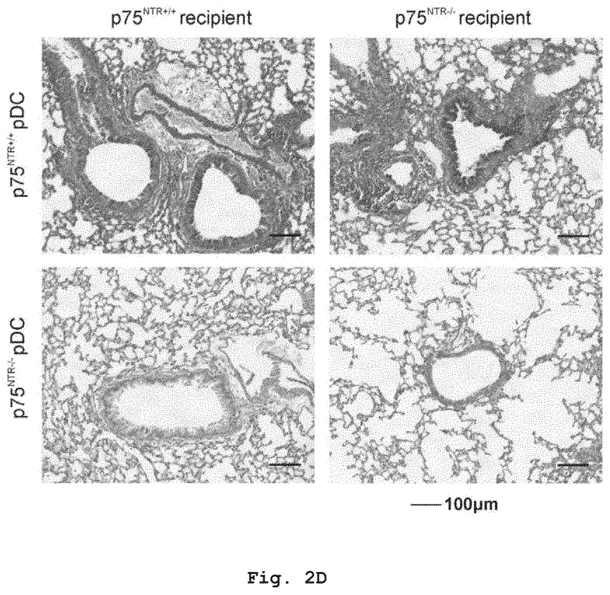

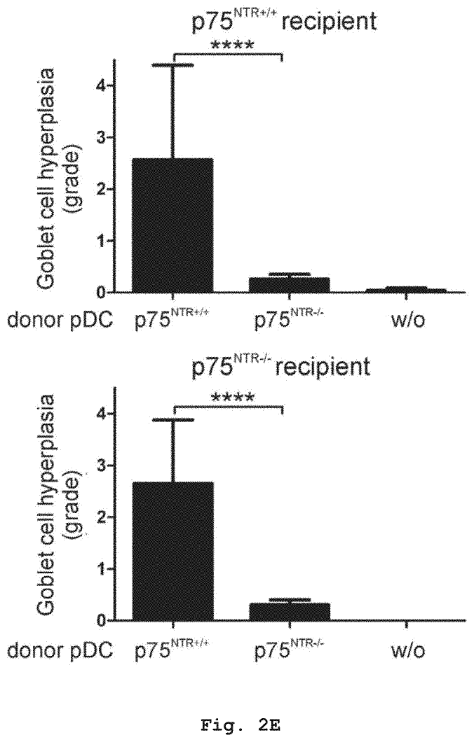

To investigate the role of p75^{NTR} expressed on PDCs in the process of disease triggering, we used the mouse model of OVA-mediated allergic asthma. OVA-pulsed PDCs from mice were intratracheally applied to the lung of either p75^{NTR+/+} or p75^{NTR-/-} mice. After provocation with OVA aerosol characteristic symptoms of asthma like severe eosinophilia, lung inflammation and intensive mucus production were analysed. p75^{NTR+/+} mice treated with OVA-loaded p75^{NTR-/-} PDCs showed significantly reduced numbers of immune cells in the BALF (lymphocytes and eosinophils) compared to mice that received p75^{NTR+/+} PDCs (FIGS. 2A, 2B). OVA-mediated immune response further lead to increased Th2 cytokine secretion (IL-4, IL-5 and IL-13) in the BALF of mice treated with p75^{NTR+/+} PDCs but not in mice that received p75^{NTR-/-} PDCs (FIG. 2C). Perivascular inflammation and GobleT-cell hyperplasia in the lung were diminished in mice treated with p75^{NTR-/-} PDCs compared to mice treated with p75^{NTR+/+} PDCs (FIGS. 2D, 2E). In summary, mice that received PDCs lacking p75^{NTR} developed significantly less allergic asthma.

It has known in the art that blocking or deleting of p75^{NTR} in mice prevented the development of lung inflammation and airway hyperresponsiveness. In the present study, however, it could be shown for the first time that allergic asthma can be induced in p75^{NTR-/-} mice by intratracheal application of p75^{NTR+/+} PDCs loaded with OVA. In contrast, application of OVA-loaded p75−/− PDCs did not induce asthma. In detail, immune cells are significantly increased in the BALF of p75^{NTR-/-} mice that received p75^{NTR+/+} PDCs (FIGS. 2A, 2B). In addition, Th2 cytokine profile (IL-4, IL-5 and IL-13) is significantly enhanced compared to mice that received p75^{NTR-/-} PDCs (FIG. 2C). Histological examination of lung tissue revealed that mice treated with p75^{NTR+/+} PDCs developed severe perivascular inflammation and Goblet-cell hyperplasia compared to mice that received p75^{NTR-/-} PDCs (FIGS. 2D, 2E).

### Example 3: NGF Regulates Interferon-Alpha (IFNα; Th1 Response) and IL-6 Production (Th2 Response) by ODN (Oligodeoxynucleotides) Stimulated Human PDC

Methods

Lymphocyte Separation

Blood samples used for cell purification were obtained from two different sources: Fresh buffy coat samples, not older than 8 hours, served as the source of PDC used in oligodeoxynucleotides (ODN) and anti FcεRIα stimulation experiments.

The blood samples were transferred to 50 ml tube and centrifuged (470 g for 30 minutes at room temperature (RT). Intermediate leukocyte layer, between the sedimented erythrocytes and upper phase thrombocytes, was taken off along with few millilitres of erythrocytes. In a fresh 50 ml tube leukocytes were diluted with 3 volumes of 1×PBS containing 2 mM EDTA and 0.5% BSA (PBS E/B). The mixture was layered carefully on the top of ficoll separation solution (Percoll separation solution, density 1.074 μg/ml, Biochrom AG) and centrifuged (1000 g without brakes for 20 minutes at RT). Erythrocytes and granulocytes sedimented to the bottom of the tube, mononuclear cells (lymphocytes) and platelets were collected in a fresh tube from the interface between the plasma layer in upper phase and sedimented erythrocytes/granulocytes. Collected cells were washed once with 50 ml PBS E/B and centrifuged (300 g for 10 minutes at RT). Supernatant was removed completely. For the depletion of platelets, following the first wash, the mononuclear cells pellet was washed twice by adding 50 ml PBS E/B and centrifugation (200 g for 15 minutes at RT). Upon centrifugation at 200 g, most of the platelets remain in the supernatant. The supernatant was discarded. Lymphocytes pellet was re-suspended in 20 ml PBS E/B. To remove cell clumps and blood clots, that might clog the MACS cell separation columns during cell purifications, cells were passed through a nylon mash having 40 μm pore size (Cell Strainer, BD Biosciences).

Plasmacytoid Dendritic Cell (PDC)/BDCA4⁺ Cell Purification

Alike CD4⁺ T helper cells isolation from peripheral blood mononuclear cells, total cell numbers were determined prior to purification of PDC. PDC were purified by using CD304 (BDCA-4/Neuropilin-1) microbead kit (Miltenyi Biotech) by positive selection, following manufacturer's instructions with some modifications. Briefly, the cell suspension was centrifuged (450 g for 6 minutes) and the pellet was re-suspended in 300 μl of PBS E/B. Then 100 μl of each FcR blocking reagent and CD304 (BDCA-4/Neuraophilin-1) microbeads were added per 10⁸ total cells. Cell suspension was incubated at 4° C. for 20 minutes. Cells were washed with 10 ml PBS E/B and centrifuged (470 g for 6 minutes). Cell pellet was re-suspended in 500 μl of PBS E/B and was loaded on rinsed MACS LS cell separation column (Miltenyi Biotech). Labelled cells were attached to the separation column. The separation column was washed three times with 1 ml PBS E/B. To increase the purity of PDC, the eluted fraction was enriched over second MACS MS cell separation column. Magnetic cell separation procedure as described for first LS column was repeated for second MS column except that washing of the MS column was carried out with 500 μl PBS E/B. Purified PDC were counted and purity was assessed by flow cytometric analysis after staining the cells with monoclonal mouse anti human BDCA2-PE (Miltenyi Biotech) and monoclonal mouse anti human CD271-APC (Miltenyi Biotech). Volume of reagents and buffers mentioned are for up to 10⁸ total cells. Whenever, higher than given total cells numbers were used the volume of reagents and buffers were also scaled-up accordingly.

IFN-Alpha Produced by Oligodeoxynucleotides (ODN) Stimulated PDC

PDC isolated from peripheral blood were taken up in RPMI 1640 medium (PAA) containing penicillin G (100 U/ml), streptomycin (100 mg/ml), L-glutamine (2 mM), 10% heat-inactivated fetal bovine serum (FBS) and Interleukin-3 (1L-3, R&D Systems) at 10 ng/ml. 5×10⁴ cells were seeded per well in 200 μl medium in U-shaped bottom 96-well plate and incubated at 5% CO₂ and 37° C. For IFNα induction, stimulatory ODN 2216 and control ODN 2243 (Alexis Biochemicals), were added at 0.33 μg/well to the designated wells. p75^{NTR} blocking peptide TAT-Pep5 (Calbiochem) was employed at 100 nM to designated wells. NGF (R&D Systems) was added at 200 ng/ml to the allocated wells. All components were added in order of succession as mentioned. After 12-14 hours stimulation the plate was centrifuged (270 g for 5 minutes). Supernatant was collected and was analyzed for IFNα quantification by ELISA (Bender MedSystems).

IFNα ELISA

IFNα ELISA was carried out according to manufacturer's instructions with slight modifications. In short, 100 μl of 10 μg/ml coating antibody in PBS was added to each of the allocated wells on flat bottom 96 well EIA/RIA stripwell plate (Corning Incorporated). Plate was covered with Parafilm M (Pechiney Plastic Packaging Company) and incubated over night at 4° C. Wells were aspirated and washed three times with washing buffer (PBS containing 0.05% Tween 20). Plate was blocked by adding 250 μl assay buffer (5 g BSA added to 1 litre washing buffer) to each well and was incubated at room temperature for 2 hours. Before adding samples, the wells were emptied and plate was washed twice with 300 μl washing buffer. 100 μl assay buffer was added in duplicate to blank wells and wells allocated for standard, leaving the first wells (500 pg/ml) empty. 90 μl assay buffer was added in duplicate to all wells designated for samples. IFN-alpha protein standard (50 ng/ml) was diluted in 500 μl assay buffer to obtain final concentration of 500 pg/ml. IFN-alpha row dilutions ranging from 500 to 8 pg/ml served as standard. 10 μl supernatant was added and mixed to the allocated wells. Horseradish Peroxidase (HRP)-conjugated detection antibody was diluted 1:1000 with assay buffer and 50 μl was added to all the wells, including blank wells. Plate was incubated at room temperature for 2 hours. The contents of wells were removed and wells were washed 3 times with 300 μl wash buffer per well. 100 μl 3,3′,5,5′-Tetramethylbenzidine (TMB) substrate solution (Sigma) was added to all wells and the plate was incubated at room temperature for 10 minutes. When dark blue colour was developed in the well with highest concentration protein standards, enzyme-substrate reaction was stopped by adding 100 μl of 4N sulphuric acid solution into each well. Absorbance of whole plate was read on spectrophotometer (TECAN, Infinite 200) at 450 nm as primary wave length and 630 nm as reference wave length.

IL-6 Produced by Anti-FcεRIα Activated PDC

PDC isolated from peripheral blood were taken in RPMI 1640 medium (PAA) with penicillin G (100 U/ml), streptomycin (100 mg/ml), L-glutamine (2 mM), 10% heat inactivated FBS and IL-3 (R&D system) at 10 ng/ml. 2×10⁵ cells were seeded per well in 200 μl medium in U-shaped bottom 96-well plate and incubated at 5% CO₂ and 37° C. For IL-6 generation, mouse anti human FcεRI alpha-FITC (eBioscience) was added at 250 ng/ml to designated wells. p75^{NTR} blocking peptide TAT-Pep5 (Calbiochem) was employed at 100 nM to designated wells. NGF (R&D Systems) was added at concentration of 25 ng/ml to specified wells. All components were added following the sequence mentioned After 14 hour's stimulation the plate was centrifuged (270 g for 5 minutes). Supernatant was analyzed for IL-6 by ELISA (Bender MedSystems).

IL-6 ELISA

IL-6 ELISA was carried out following manufacturer's specifications with few modifications. In short, 100 μl of 2.5 μg/ml coating antibody in PBS was added to each of the allocated wells on flat bottom 96 well EIA/RIA stripwell plate (Corning Incorporated). Plate was covered with Parafilm M (Pechiney Plastic Packaging Company) and incubated over night at 4° C. Wells were aspirated and washed three time with 300 μl washing buffer (PBS containing 0.0005% Tween 20) per well. Plate was blocked by adding 250 μl assay buffer (washing buffer containing 0.005% BSA) to each well and plate was incubated at room temperature for 2 hours. Before adding samples the wells were emptied and plate was washed twice with 300 μl washing buffer. 100 μl assay buffer was added in duplicate to blank wells and wells allocated for standard. 60 μl assay buffer was added in duplicate to all the wells designated for samples. 2 ng/ml IL-6 standard proteins was diluted in 250 μl assay buffer to obtain final concentration of 200 pg/ml. Serial dilutions of IL-6 protein ranging from 100 to 1.6 pg/ml served as standard. 40 μl supernatant was added and mixed to the wells allocated for samples. Biotin-conjugated detection antibody was diluted 1:1000 with assay buffer and 50 μl was added to all the wells. Plate was incubated at room temperature for 2 hours. The contents of wells were removed and swashed 3 times with 300 μl of wash buffer per well. 100 μl of streptavidin-HRP, 1:5000 diluted with assay buffer, was added to all the wells and the plate was incubated at room temperature for an hour. Wells were aspirated and were washed 3 times with 300 μl wash buffer per well. 100 μl TMB substrate solution (Sigma) was added to all wells, including blank wells and the plate was incubated in dark at room temperature for 10 minutes. Enzyme-substrate reaction was stopped by adding 100 μl of 4N sulphuric acid, into each well before positive wells were no longer properly recordable. Absorbance of whole plate was read on spectrophotometer at 450 nm as primary wave length and 620 nm as reference wave length.

### Example

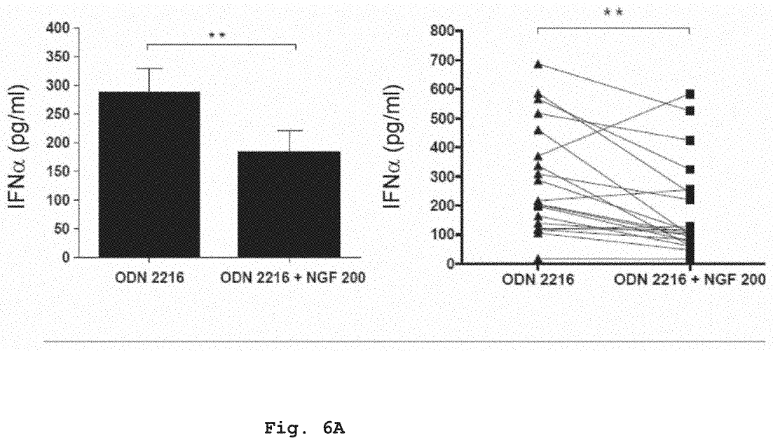

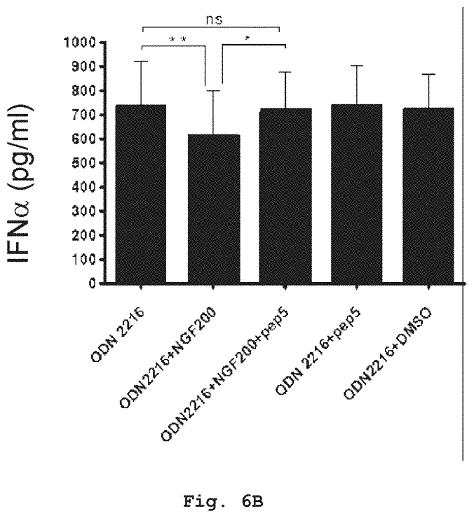

Human PDC express the Toll-like receptor 9 (TLR9). Stimulatory ODN 2216 (A-Class CpG ODN) are recognized by TLR9 expressed by PDC. Recognition of ODN 2216 by TLR9 activates PDC and induces anti-viral IFNα secretion (Th1 response). We stimulated human peripheral blood purified PDC with ODN 2216 plus minus NGF at 200 ng/ml. After 14 hours of stimulation the supernatant was collected and analyzed for IFNα secretion by ELISA. We have used 20 samples. Our results revealed significant reduction (p=0.0031) in IFNα secretion by ODN 2216 plus NGF 200 activated PDC compared to PDC activated with ODN 2216 without NGF (FIG. 6A). Control ODN 2243 did not stimulate PDC at all, thus no IFN-α was detected in supernatant (data not shown). To prove that regulation of IFNα secretion in ODN 2216 activated PDC by NGF is through CD271 and not through TrkA, we used TAT-Pep 5 (p75^{NTR} signalling inhibitor) to rescue the NGF mediated, reduced IFNα production by ODN 2216 activated PDC. In total 23 buffy coat samples were analyzed. The NGF dependent, decrease in IFNα production was significantly rescued by addition of TAT-Pep5 (100 nM; p=0.0168). TAT-Pep5 by itself and/or DMSO (solvent for TAT-Pep5) did not alter IFNα secretion in ODN 2216 activated PDC compared to ODN 2216 activated PDC without NGF (FIG. 6B).

Furthermore, PDC are reported to express FcεRIα, the high affinity receptor for IgE. We have used anti-FcεRIα-FITC, a cross linker to IgE high affinity receptor, to stimulate PDC. Upon IgE receptor cross linking PDC becomes activated hence Th2 response is triggered by secretion of proinflammatory cytokine IL-6.

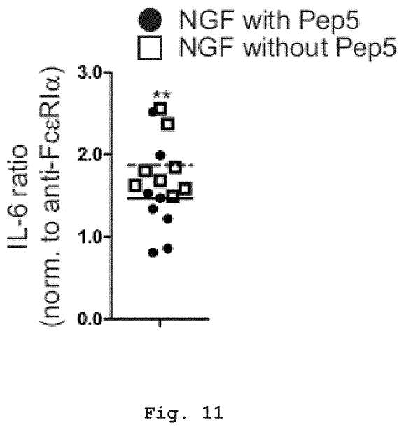

We activated peripheral blood purified PDC by cross linking FcεRIα with anti-FcεRIα-FITC in the presence and absence of NGF at 25 ng/ml in-vitro. Supernatant was harvested after 12 hrs of activation and amount of IL-6 secreted was determined by ELISA. We have analysed 18 buffy coat samples. Our results demonstrated that addition of NGF at 25 ng/ml has significantly increased (p=0.0066) the production of IL-6 from PDC activated by cross linking of IgE high affinity receptor compared with PDC activated with IgE cross linker without NGF (FIG. 11). The NGF dependent, increase in IL-6 production was significantly reduced by addition of TAT-Pep5 (100 nM; FIG. 11).

### Example 4: NGF Promotes Antigen Mediated Proliferation of Human CD4⁺ T-Cells

Methods

CD4+ T Helper Cell Purification

CD4⁺ T helper cells and PDC used in antigen mediated autologous CD4⁺ T-cell proliferation assays were purified from peripheral blood (80 ml) obtained from specified donors who are known to have allergy against certain allergens. Peripheral blood was drawn in tubes containing Li-Heparin as anti-coagulant (S-Monovette Li-Heparin 7.5 ml, Sarstedt). CD4⁺ T helper cells were purified from peripheral blood mononuclear cells by negative selection using CD4⁺ T-cell isolation kit II (Miltenyi Biotec), as per manufacturer's instructions with slight modifications. Briefly, the cell pellet was re-suspended in 50 μl PBS E/B. Then 12 μl of Biotin-labelled antibody cocktail per 10⁷ total cells were added and the cell suspension was incubated at 4° C. for 15 minutes. 50 μl of PBS E/B was added followed by addition of 25 μl of anti-biotin microbeads per 10⁷ total cells. Cells were incubated at 4° C. for another 20 minutes, followed by washing with 10 ml PBS E/B and centrifugation (470 g for 6 minutes). Supernatant was taken off completely and pellet was re-suspended in 500 μl of PBS E/B. Cell suspension was applied to equilibrated MACS MS separation column and enriched CD4⁺ T helper cells fraction was obtained in the flow through. The purity of isolated CD4⁺ T helper cells was over 95% as assessed by flow cytometric analysis after co-staining with monoclonal mouse anti human CD3-PE (BD Biosciences) and monoclonal mouse anti human CD4-FITC (BD Biosciences). When more than 10⁷ cells were used volumes of reagents and buffer were scaled-up, accordingly.

Carboxyfluorescein Succinimidyl Ester (CFSE) Labelling of CD4 T Helper Cells

Purified CD4⁺ T helper cells (see above) were washed once with PBS. 3-8×10⁶ cells were re-suspended in 1 ml PBS containing 5% BSA. One aliquot of CFSE powder (Molecular Probes, Invitrogen Technologies) was dissolved in 18 μl DMSO to obtain final concentration of 5 mM. CD4⁺ T-cells were stained with a 1 μM final concentration of CFSE by incubating the cell suspension plus 1 μM CFSE at 37° C. for 8 minutes. 1 ml pre-warmed FCS was added to the suspension and the cells were washed with RPMI medium twice. Cell number was determined.

Co-Culture of Autologous PDC and CFSE Labelled CD4⁺ T Helper Cells

Antigen mediated CD4⁺ T helper cell proliferation responses were assayed using purified and CFSE labelled CD4⁺ T helper cells in co-culture with purified PDC/BDAC4⁺ cells. PDC and T-cells were re-suspended separately in RPMI-1640 medium supplemented with penicillin G (100 U/ml), streptomycin (100 mg/ml), L-glutamine (2 mM) and 10% human AB serum. The ratio of PDC to T-cell in co-culture was kept 1:6. Antigens were added to co-culture at 50 SBE U/ml. NGF was added at the concentration of 5, 25 and 50 ng/ml. The assay was set up in U-bottom 96-well plate at 37° C. in 5% CO₂. After 5 days of co-culture, supernatant was analyzed for Th1/Th2 cytokines by using BD cytokine bead array (CBA) Human Th1/Th2 cytokine kit (BD Biosciences) and percentages of proliferating CFSE-low CD4⁺ T-cells were analyzed by flow cytometry.

Cytokine Measurement by Cytokine Bead Array (CBA)

The concentrations of IL-2, IL-4, IL-5, IL-10, IFNγ and TNFα in the supernatant were determined by using the CBA kit following manufacturer's instruction with modification in data analysis using Microsoft Excel. Briefly, a CBA comprises beads exhibiting series of discrete fluorescence intensities that is resolved in FL3 channel of a flow cytometer. Each series of beads is coated with a monoclonal antibody specific for a single cytokine, and a mixture of six beads can detect six different cytokines in a single sample. The PE-conjugated detection antibody stains the beads proportionally to the amount of cytokine bound. After fluorescence intensity calibration and colour compensation procedures, standards and test sample (supernatant) were analyzed with FACS LSRII flow cytometer equipped with DIVA software (BD Biosciences). The standard curve created for each cytokine was used to calculate the cytokine concentration. The lower detection limits for IL-2, IL-4, IL-5, IL-10, TNFα and IFNγ were 2.6 pg/ml, 2.6 pg/ml, 2.4 pg/ml, 2.8 pg/ml, 2.8 pg/ml, and 7.1 pg/ml, respectively.

### Example

PDCs are professional antigen presenting cells. PDCs were purified using BDCA4⁺ microbeads and CD4⁺ T-cells by negative selection from peripheral blood of patients with allergy against certain known allergens such as grass antigen and guinea pig antigen. Purified and CFSE (carboxyflourescein succinimidyl ester) labelled CD4⁺ T-cells were co-cultured in duplicates with autologous PDCs in the presence of optimal concentration of allergy specific allergen/antigen with and without NGF at 5, 25 and 100 ng/ml. After 5 days in culture the proliferation of CD4⁺ T-cells was determined by diminishing CFSE fluorescence. As shown in FIG. 7A, NGF at 25 ng/ml demonstrated significantly increased antigens (allergen) mediated CD4⁺ T-cells proliferation compared with autologous CD4⁺ T-cell/PDC co-cultured without NGF. In contrast, with control antigen very little proliferation was noticed. T-cells on its own (without PDC co-culture) did not show any proliferation whether incubated with or without antigen (allergen) plus minus NGF or with NGF alone. Furthermore, NGF induced a dose-dependent increased secretion of pro-inflammatory Th2 cytokines IL-2 and IL-5 (FIG. 7B) compared with autologous CD4⁺ T-cell/PDC co-cultured without NGF.

### Example 5: NGF Controls Cytokine Secretion and TLR Signalling of Murine PDC in a p75^{NTR} Dependent Manner

Methods

as described above

### Example

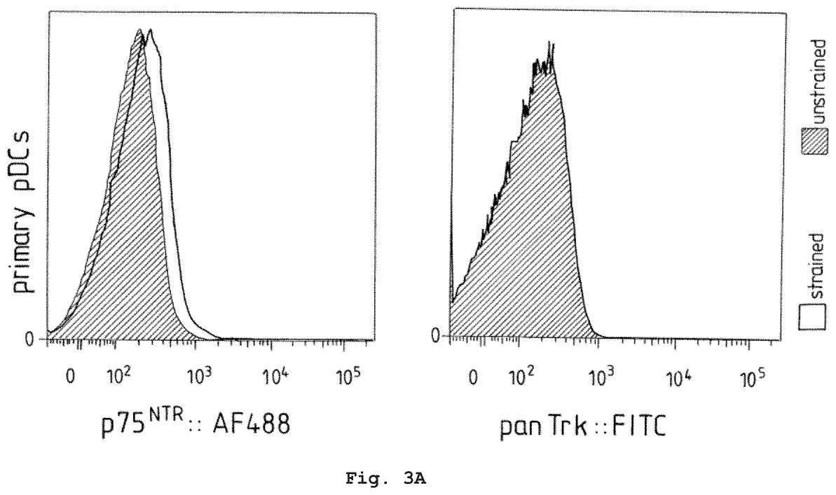

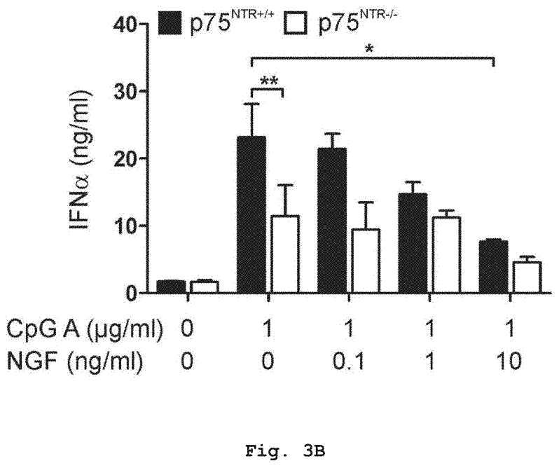

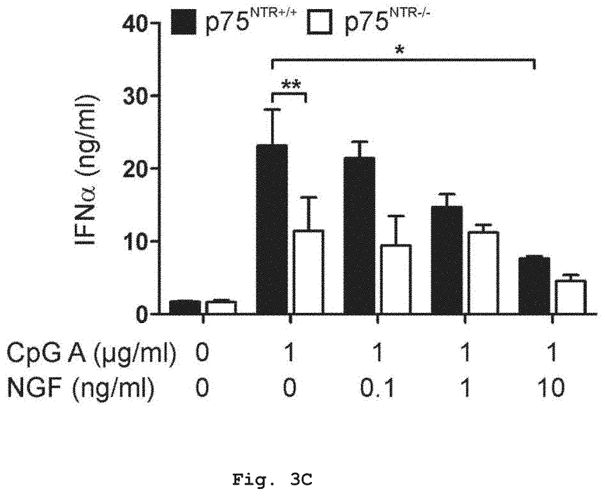

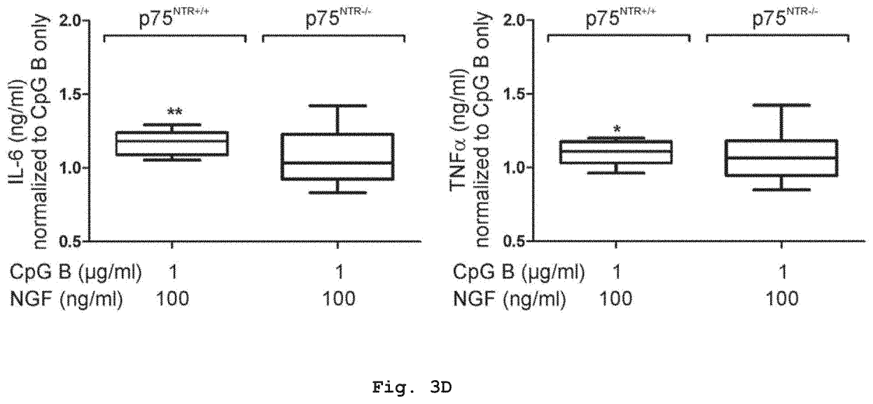

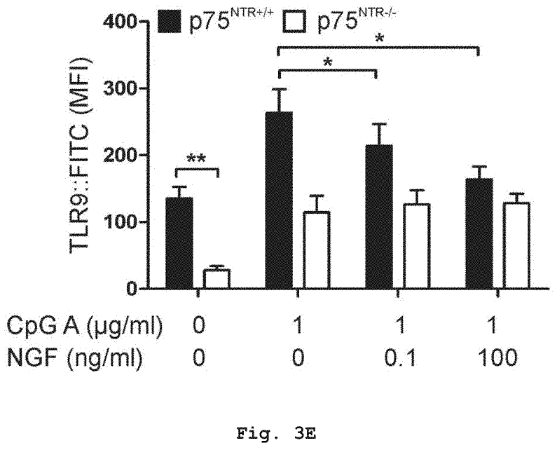

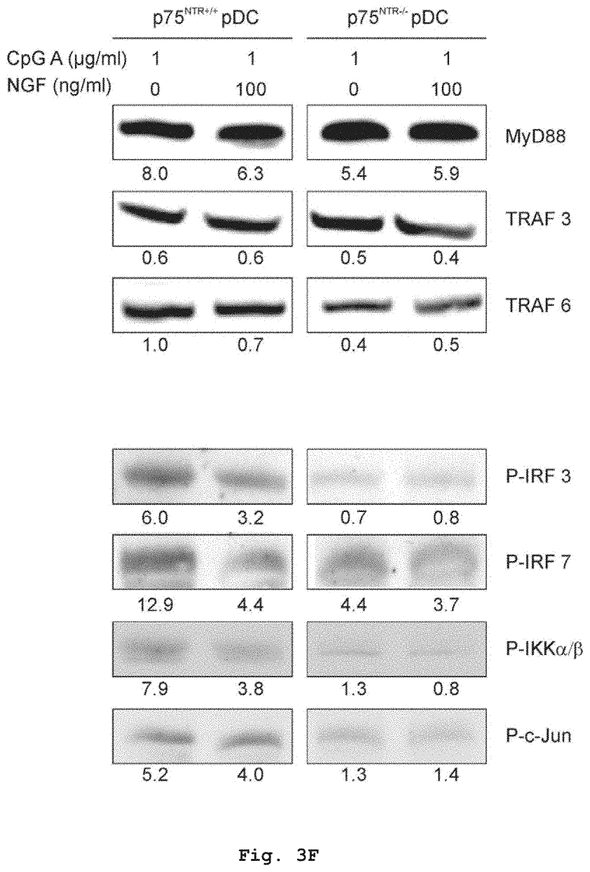

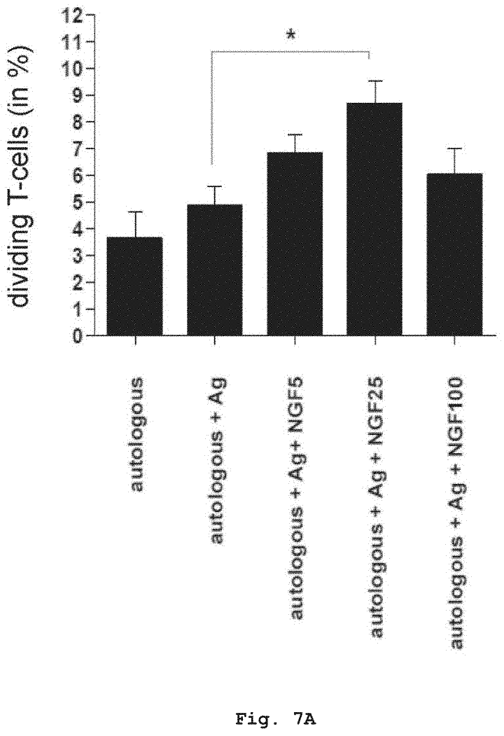

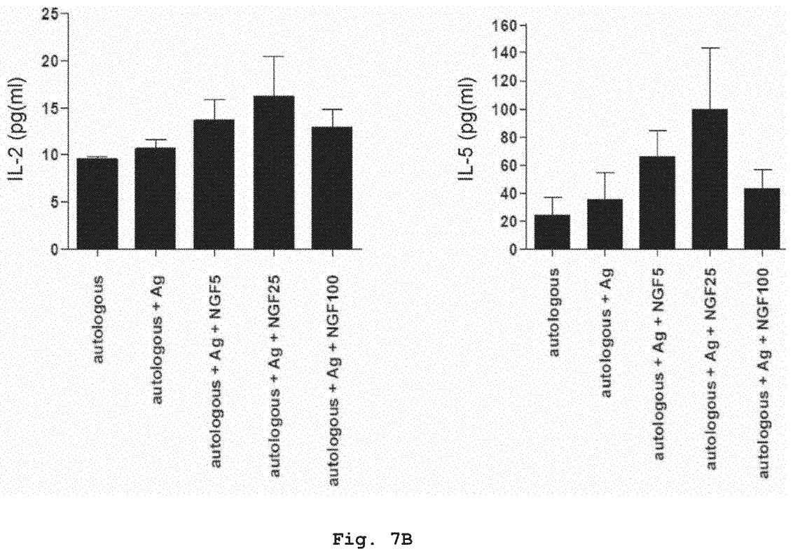

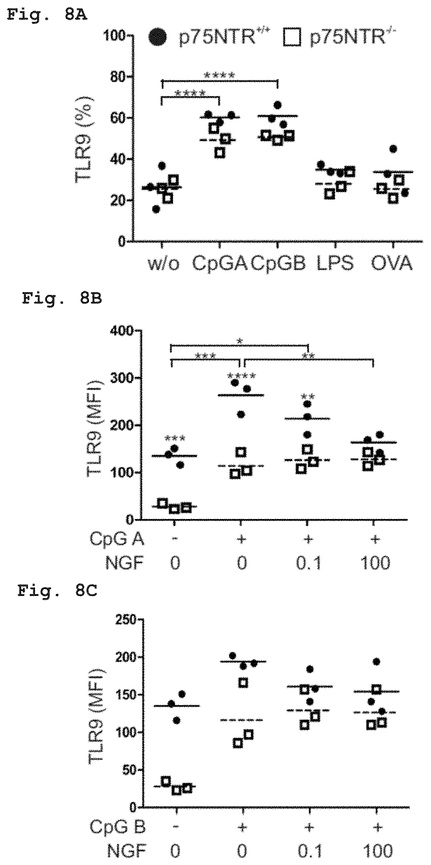

Murine PDC express the low affinity neurotrophin receptor p75^{NTR} but not the high affinity neurotrophin receptors (FIGS. 3A, 3B). CPG-ODNs Class A stimulated PDCs secrete decreasing levels of IFNα (FIG. 3C) and display reduced expression of TLR9 (FIGS. 3E, 8A) in the presence of increasing NGF levels. PDCs of p75^{NTR} knockout mice (p75^{NTR-/-}) does not display a NGF induced alteration of TLR9 expression upon CPG-ODNs Class A or Class B stimulation (FIGS. 8B, 8C). Furthermore, only p75^{NTR} expressing PDC (p75^{NTR+/+}) increased the CPG-ODNs Class B induced secretion of the Th2 response inducing cytokines IL-6 and TNFα in the presence of NGF (FIG. 3D).

To investigate the mechanisms underlying these effects, we analysed PDCs treated with CPG-ODNs Class A (FIG. 3F) or Class B (FIG. 3G) using western blotting. P75^{NTR+/+} and p75^{NTR-/-} PDCs expressed comparable levels of MyD88, TRAF3 and TRAF6, which are involved in signalling pathways activated by CpG A and CpG B. NGF attenuated CpG A-induced phosphorylation of the transcription factors IRF3 and IRF7 in p75^{NTR+/+} PDCs, while CpG B increased phosphorylation of IRF3 and IRF7. NGF did not alter the levels of CpG-induced phosphorylation of IRF3 and IRF7 expressed by p75^{NTR-/-} pDCs.

### Example 6: NGF Controls Expression of Co-Stimulatory and Antigen-Presenting Molecules, and Cytokines by Murine PDCs in a p75^{NTR} Dependent Manner

Methods

as described above

### Example

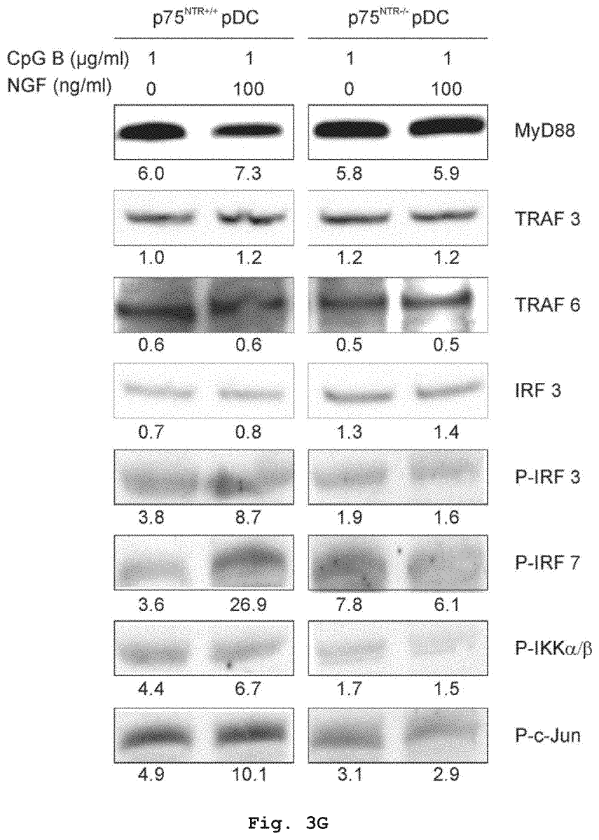

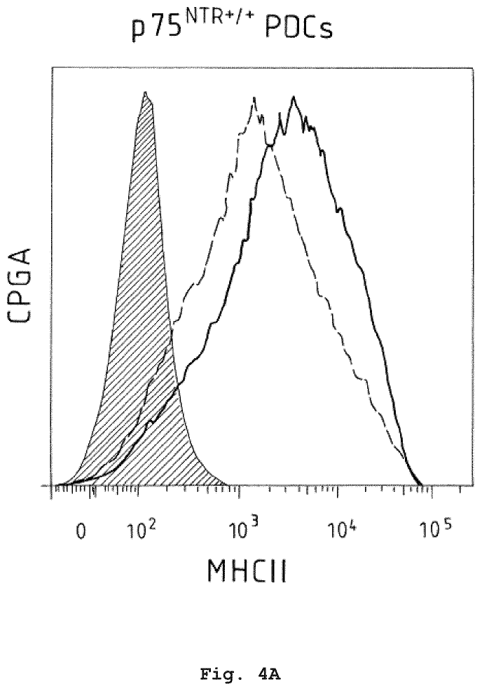

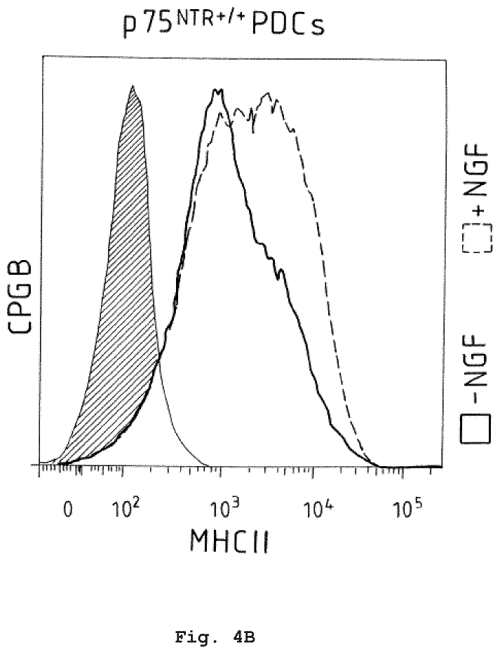

Upon stimulation with the Th1-priming CPG-ODNs Class A murine p75^{NTR+/+} PDCs displayed a reduced expression of the CD4 T-cell specific, antigen-presentation molecule MHC-II in the presence of NGF (FIG. 4A), whereas p75^{NTR+/+} PDCs stimulated with the Th2-priming CPG-ODNs Class B displayed an increased expression of the antigen-presentation molecules MHC-II (FIG. 4B) and the CTL-specific MHC I (FIG. 4C) in the presence of NGF.

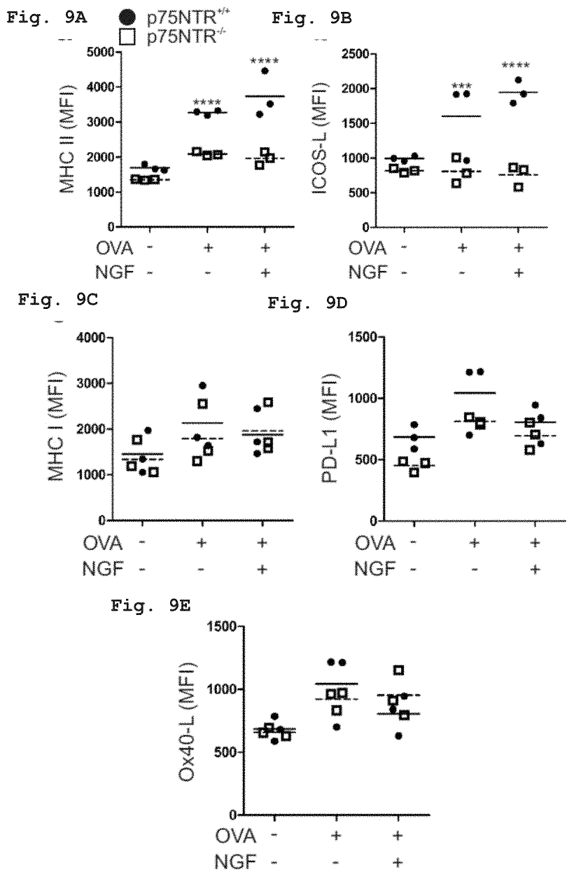

Upon stimulation of murine PDCs with TLR7 and TLR9 ligand containing ovalbumin, p75^{NTR+/+} PDCs displayed a significantly increased expression of antigen-presentation and co-stimulatory molecules (ICOS-L, MHC-II) which was only further increased by addition of NGF to p75^{NTR+/+} PDCs, whereas p75^{NTR-/-} PDCs did not (FIGS. 9A, 9B). Furthermore, addition of NGF altered the expression of MHC-I, PD-L1 and Ox40-Land driving MHC-II molecule (MHC-II) on p75^{NTR+/+} PDCs, but not on p75^{NTR-/-} PDCs (FIGS. 9C-9E).

### Example 7: NGF Reduces PDC Dependent Secretion of Th1 Cytokines IL-2 and IFNγ by Murine T-Cells

Methods

as described above

Isolation of Murine T-Cells

Murine T-cells were isolated from the OTII mouse strain expressing mouse alpha-chain and beta-chain T-cell receptor specific for chicken ovalbumin 323-339, using the CD+ T-cell isolation kit (Miltenyi Biotec).

### Example

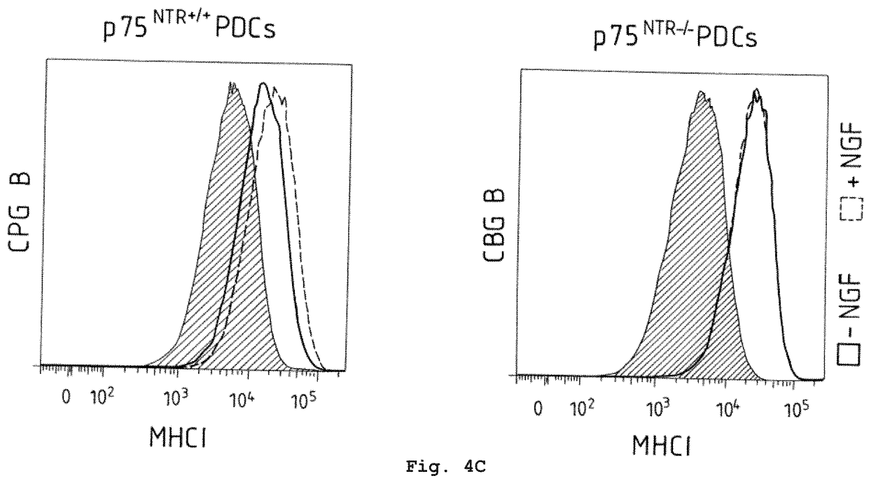

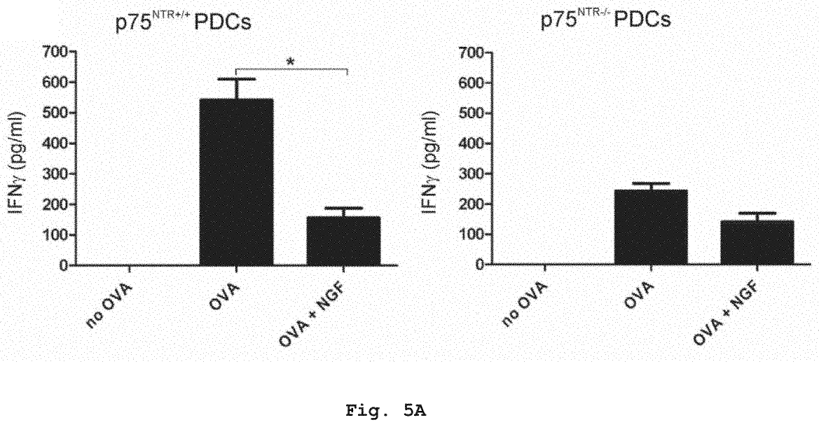

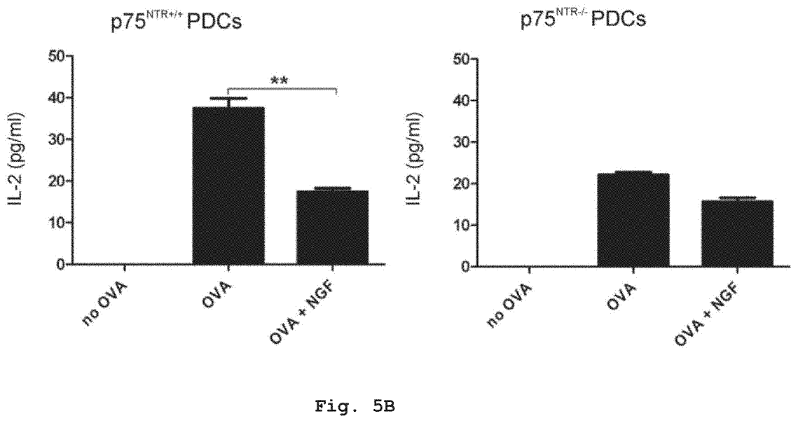

Both, murine PDCs and T-cells were cultured overnight with or without the chicken ovalbumin peptide 323-339 in the presence or absence of NGF (100 ng/ml). The concentrations of T-cell secreted Th1-cytokines IL-2 and IFNγ in the supernatant were determined by using the CBA kit (BD Bioscience) following manufacturer's instruction with modification in data analysis using Microsoft Excel. FIG. 5 shows the influence of NGF on the secretion of the Th1 cytokines IFNγ and IL-2 in the co-culture. In the presence of p75^{NTR+/+} PDCs presenting the ovalbumin peptide (OVA) to the T-cells, T-cells secrete the Th1 cytokines IFNγ (FIG. 5A) and IL-2 (FIG. 5B). Compared to co-culture with p75^{NTR-/-} PDCs, T-cells co-cultured with PDCs from the p75^{NTR+/+} strain react with reduced secretion of both Th1 cytokines upon addition of NGF.

### Example 8: NGF Controls PDC Induced Proliferation and Cytokine Secretion of Murine T-Cells in a p75^{NTR} Dependent Manner

Methods

as described above

Isolation of Murine T-Cells

Murine T-cells were isolated either from OT-II mouse strain expressing ovalbumin peptide specific T-cell receptors on CD4+ T-cells or from OT-I mouse strain expressing ovalbumin peptide specific T-cell receptors on CD8+CTLs using the CD8+ T-cell isolation kit (Miltenyi Biotec).

### Example

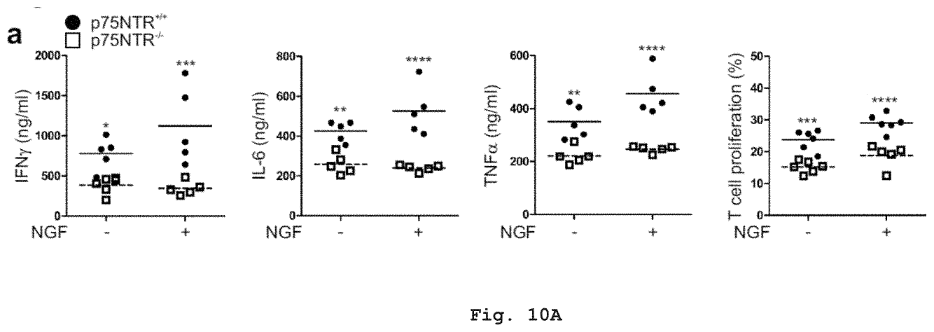

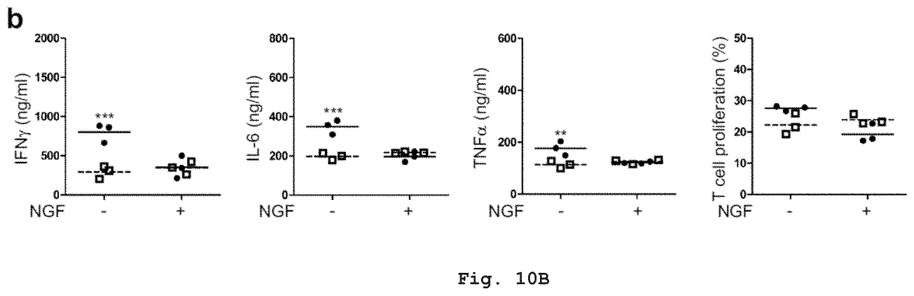

Compared to co-culture with p75^{NTR-/-} PDCs, CD4+ T-cells from OT-II strain co-cultured with PDCs from the p75^{NTR+/+} strain react with increased Th2 cytokine secretion and proliferation upon addition of NGF (FIG. 10A), whereas CD8+CTLs from OT-I strain secreted less cytokines and showed reduced proliferation when NGF was present in co-culture (FIG. 10B).

### Example 8: NGF Aggravates Th2 Prone GvHD in Xenotransplantation Model

Methods

As described above

Mouse Strain

Recipient mice from NSG mouse strain were pre-condition by irradiation with 3 Gy 24 hours before transplantation of human cells

GvHD Scoring and Survival

For scoring of GvHD incidence and survival transplanted mice were monitored daily for weight, behaviour, skin, activity, fur and other parameters.

### Example

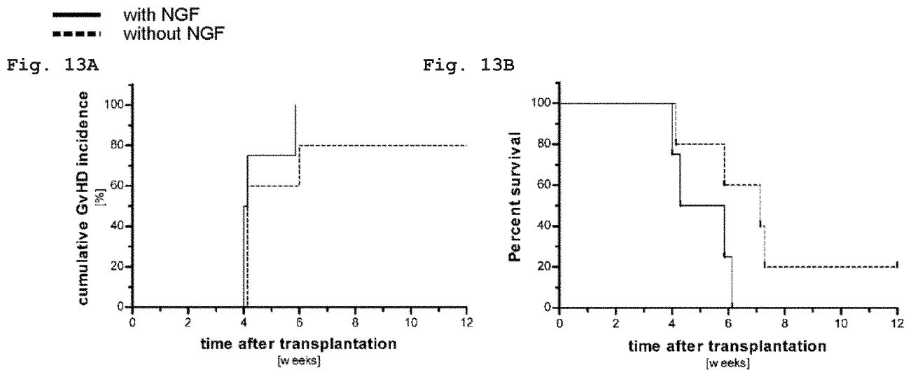

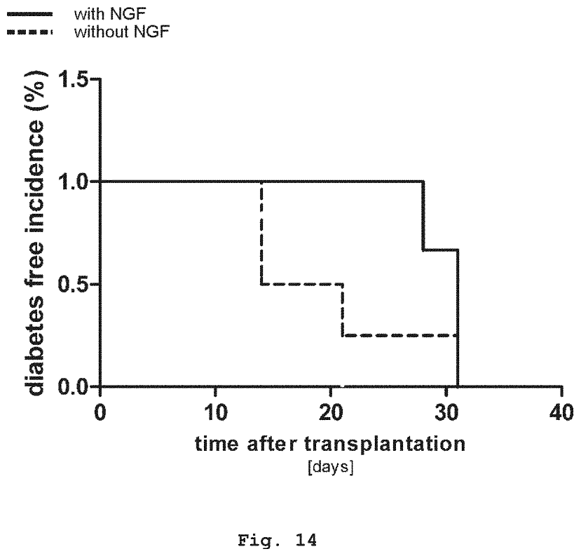

Human, autologous T-cells and PDCs were stimulated either with CpG B or not. Overnight co-cultured was done in the absence or presence of NGF. One day later human cells were transplanted into irradiated NSG mice by i.v. injection. Over a time period of 12 weeks post transplantation an increased severity of GvHD (cumulative GvHD incidence, FIG. 13A) could be observed, when PDCs were co-cultured in the presence of NGF underlining a superstimulation of Th2 T-cell response. In addition, a significantly increased mortality of these mice was observed (FIG. 13B). When PDCs were cultured without TLR stimulating CpG B, no NGF dependent effect on cumulative GvHD incidence or survival could be observed (data not shown).

### Example 9: NGF Alleviates Th1 Prone Type I Diabetes in Mice

Methods

As described above

Mouse Strain

For the applied type I diabetes mouse model the RIP-CD80× RIP-LMV-GP mouse strain was used. This strain over-expresses the co-stimulatory CD80 molecule to enhance T-cell response. Furthermore, a glycoprotein of the LCMV virus is expressed to enable artificial targeting of the pancreatic beta-cells to initiate type I diabetes.

Initiation and Assessment of Type I Diabetes

Murine PDCs were cultured for one hour together with the LCMV glycoprotein in the absence or presence of NGF. Subsequently, PDCs were injected i.v. into the recipient mice. Two weeks after transplantation mice were observed thrice a week for blood glucose level. With a consecutive blood glucose level above 250 mg/l mice were diagnosed as diabetic.

### Example

Murine PDCs were stimulated with CpG B and co-incubated with the LCMV glycoprotein. In the presence of NGF in the culture, PDC induced type I diabetes occurred at a significant later stage than in mice transplanted with PDCs cultured in the absence of NGF. When PDCs were cultured without TLR stimulating CpG B, no NGF dependent effect on type I diabetes incidence could be observed (data not shown).

1. A combination comprising at least one modulator of p75^{NTR} signalling selected from a p75^{NTR} signalling antagonist or p75^{NTR} signalling agonist and at least one agonist of TLR7 and/or TLR9.

2. A vaccine composition comprising the combination of claim 1.

3. The combination according to claim 1, wherein said p75^{NTR} signalling agonist is selected from
i) NGF, BDNF, NT-3, NT-4, and NT-5;
ii) activating antibodies;
iii) activating peptides and activating small molecules;
iv) activating peptides; or
v) a nucleic acid.

4. The combination according to claim 1, wherein said antagonist of p75^{NTR} signalling is selected from
i) pro-NGF, pro-BDNF, pro-NT-3, pro-NT-4, and pro-NT-5;
ii) blocking antibodies, derivatives and humanized versions thereof; anti mouse p75^{NTR} monoclonal antibody;
iii) antibodies that prevent binding of neurotrophins to p75^{NTR}, derivatives and humanized versions thereof;
iv) blocking peptides;
v) peptides that block the interaction of p75^{NTR} with TRAF6;
vi) blocking proteins that prevent binding of neurotrophins to p75^{NTR};
vii) small molecule inhibitors, small molecules that prevent binding of neurotrophins to p75^{NTR};
viii) morpholinos that block expression of p75^{N}m; or
ix) a nucleic acid that blocks expression of p75^{NTR} or downstream signalling.

5. The combination according to claim 1, wherein said agonist of TLR7 and/or TLR9 is selected from:
i. TLR7 agonists selected from single stranded RNAs, CL075, CL097, CL264, CL307, Gardiquimod, Imiquimod, Loxoribine, poly(dU), poly(dT), R848 and IMO-4200;
ii. TLR9 agonists selected from bacterial DNA and CPG-ODNs Class A;
iii. Dual agonists of TLR7 and TLR9;
iv. Live or attenuated viruses, bacteria, parasites;
v. Viral, bacterial or parasitic extracts.

6. The vaccine composition according to claim 2, further comprising at least one immune stimulating agent which is selected from monophosphoryl lipid A (MPL) and synthetic derivatives thereof, muramyl dipeptide (MDP) and derivatives thereof, oligodeoxynucleotides, double-stranded RNA (dsRNA), alternative pathogen-associated molecular patterns (PAMPs), saponins, small-molecule immune potentiators, cytokines, chemokines and antigens from Mycobacterium tuberculosis.

7. The vaccine composition according to claim 6, further comprising at least one agent selected from insoluble aluminium compounds, calcium phosphate, liposomes, virosomes, immune stimulating complexes (ISCOMS), microparticles, emulsions, virus-like particles and viral vectors.

8. The vaccine composition according to claim 6, further comprising isolated p75^{NTR} expressing PDCs, in vitro generated p75^{NTR} expressing PDCs, or a p75^{NTR} expressing PDC cell line.

9. A method of treatment for a patient suffering from a disease selected from the group consisting of central and peripheral neurodegenerative diseases, senile dementia, epilepsy, Alzheimer's disease, Parkinson's disease, Huntington's disease, Down's syndrome, prion diseases, amnesia, schizophrenia, depression, bipolar disorder, amyotrophic lateral sclerosis, multiple sclerosis, cardiovascular conditions, post-ischemic cardiac damage, cardiomyopathies, myocardial infarction, heart failure, cardiac ischemia, cerebral infarction, peripheral neuropathies, damage to the optic nerve and/or to the retina, retinal pigment degeneration, glaucoma, retinal ischemia, macular degeneration, spinal cord traumas, cranial traumas, atherosclerosis, stenosis, wound healing disorders, alopecia, any type of cancer, any type of tumours, any type of metastases, any type of leukemia, respiratory disorders, pulmonary inflammation, allergy, anaphylaxis, asthma, atopic dermatitis, chronic obstructive pulmonary disease, cutaneous pain, somatic pain, visceral pain, neurological pain, chronic neuropathic pain, inflammatory pain, autoimmune diseases, rheumatoid arthritis (polyarthritis, oligoarthritis), ankylosing spondylitis, collagenosis, systemic lupus erythematodes (SLE), SHARP syndrome, Sjögren's syndrome, scleroderma, polymyositis, dermatomyositis, progressive systemic sclerosis, spondyloarthritis (Morbus Bechterew, reactive arthritis, enteropathic arthritis, psoriatic arthritis, undifferentiated spondyloarthritis), rheumatic fever, Aicardi-Goutieres syndrome, vasculitis, Wegener's granulomatosis disease, nephritis, stroke, ulcerative colitis, Crohn's diesease, Morbus Whipple, scleroderma, Still's disease, bronchopulmonary dysplasia (BPD), bronchiolitis, RSV-associated bronchiolitis, Diabetes mellitus, fibromyalgia syndrome, coeliac disease, Hashimoto's disease, hypothyroidism, hyperthyroidism, Addison's disease, graft versus host disease (GVHD), autoimmune thrombocytopenia, autoimmune hemolytic anemia, Löfgren syndrome, Behcet disease, nephrotic syndrome, uveitis, psoriatic arthritis, psoriasis (plaque psoriasis, pustular psoriasis), bone fractures, bone diseases, osteoporosis and all bacterial, fungal, viral infectious diseases, as well infections with eukaryotic parasites which comprises administering to the patient an effective amount of the composition of claim 2.

10. A screening method for agonists and antagonists of p75^{NTR} signalling comprising the steps of:
Contacting primary or in vitro generated human or animal plasmacytoid dendritic cells (PDCs), or PDCs cell lines that express the nerve growth factor receptor p75^{NTR} with a test substance;
Incubating said contacted human or animal primary PDCs or PDCs cell lines for a period of time, which is sufficient for effecting p75^{NTR} signalling;
Determining the effect of the test substance on the primary or in vitro generated human or animal PDCs or PDCs cell lines;
Comparing the effect of the test substance in the contacted primary or in vitro generated human or animal PDCs or PDCs cell lines with control cells or cell lines; and
Selecting a test substance that agonizes or antagonizes p75^{NTR} signalling in primary or in vitro generated human or animal PDCs or PDCs cell lines.

11. The screening method of claim 10, wherein the human or animal PDCs or PDCs cell lines express the nerve growth factor receptor p75^{NTR} and/or at least one protein selected from the group of Toll like receptors, preferably TLR7 or TLR9.

12. The screening method of claim 10, wherein the human or animal PDCs are transgenic cells or cell lines which have been genetically modified to overexpress p75^{NTR} and/or at least one protein selected from the group consisting of TLR9, TLR7, TRAF3 and TRAF6.

13. The screening method according to claim 10, wherein the control cells or cell lines are human or animal primary cells, cells which do not naturally express p75^{NTR}, cells in which p75^{NTR} is knocked out, cells in which the expression of p75^{NTR} is reduced or inhibited, or cells in which p75^{NTR} signalling is blocked, inhibited or reduced.

14. The screening method according to claim 10, wherein the PDCs or PDCs cell lines that express p75^{NTR} are co-incubated with T-cells, comprising the steps of:
Contacting human or animal PDCs or PDCs cells or PDCs cell lines and that express the nerve growth factor receptor p75^{NTR}, which are co-incubated with T-cells, with a test substance;
Incubating said contacted co-culture of said human or animal PDCs or PDCs cell lines and said T-cells for a period of time sufficient for effecting p75^{NTR} signalling;
Determining the effect of the test substance on the PDCs or PDCs cell lines and/or on the T-cells;
Comparing of the effect of the test substance in the contacted PDCs or PDCs cell lines and/or T-cells with control cells or cell lines and/or T-cells; and
Selecting a test substance that agonizes or antagonizes p75^{NTR} signalling.

15. The screening method according to claim 10, wherein the step of contacting a human or animal PDCs or PDCs cell lines that express the nerve growth factor receptor p75^{NTR} with said test substance is performed in the presence of a natural or artificial ligand of p75^{NTR} under conditions allowing the interaction of the test substance and the p75^{NTR} protein and/or the interaction of the test substance with the natural ligand of p75^{NTR}.

16. The screening method according to claim 10, wherein the PDCs or cells or PDCs cell lines are pre-activated prior to or during their use in the screening method, suitably with at least one agonist of Toll like receptor signalling, preferably an agonist of TLR7 and/or TLR9.

17. The screening method according to claim 10, wherein antagonistic or agonistic effect of the test substance on the p75^{NTR} signalling in the assay is measured based on expression analysis of cytokines and/or analysis of intracellular signalling cascades and/or surface marker expression analysis and/or measurement of the uptake, intracellular processing and presentation of external antigens and/or analysis of T-cells.

18. The screening method according to claim 10, wherein said method is performed in vivo, characterized in that the PDCs or PDCs cell lines which express p75^{NTR} and/or at least one Toll like receptor are administered to an animal model which is specific for an immune, inflammatory or proliferative disease.

19. The screening method of claim 18, wherein determination of antagonistic or agonistic effect of a test substance in said animal models is performed in the presence of control animals which comprise at least the PDCs but in which p75^{NTR} is not expressed or expressed at lower levels, or wherein the applied PDCs or PDCs cell lines exhibit reduced or inhibited expression of p75^{NTR}, or blocked, inhibited or reduced p75^{NTR} signalling.

20. The combination of claim 1 wherein the p75^{NTR} signalling antagonist is pro-NGF, the TLR9 agonist is CPG-ODNs Class A, and further including alternative pathogen-associated molecular patterns (PAMPs).


SEQUENCE LISTING (16 sequences)

SEQ ID NO: 1 - protein, 15 aa, Homo sapiens
EGEKLHSDSGISVDS

SEQ ID NO: 2 - protein, 9 aa, Homo sapiens
RPTIPRNPK

SEQ ID NO: 3 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 4 - DNA, 3420 bp, Homo sapiens
agagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgtgatgaagaaaagtgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttgggggcttgcaagtatgttttagcatgtgtttggttctggggcccctttttactccccttgagctgagatggaacccttttggcccccgagctgggggccatgagctccagacccccagcaaccctcctatcacctcccctccttgcctcctgtgtaatcatttcttgggccctcctgaaacttacacacaaaacgttaagtgatgaacattaaatagcaaagaaagaaaaatagtacaaagag

SEQ ID NO: 5 - DNA, 20 bp, Artificial sequence
  Synthetic oligonucleotide
ggggtcaacgttgagggggg

SEQ ID NO: 6 - DNA, 20 bp, Artificial sequence
  Synthetic oligonucleotide
gggggacgatcgtcgggggg

SEQ ID NO: 7 - DNA, 21 bp, Artificial sequence
  Synthetic oligonucleotide
ggggacgacgtcgtggggggg

SEQ ID NO: 8 - DNA, 23 bp, Artificial sequence
  Synthetic oligonucleotide
tcgacgttcgtcgttcgtcgttc

SEQ ID NO: 9 - DNA, 26 bp, Artificial sequence
  Synthetic oligonucleotide
tcgcgacgttcgcccgacgttcggta

SEQ ID NO: 10 - DNA, 20 bp, Artificial sequence
  Synthetic oligonucleotide
tccatgacgttcctgatgct

SEQ ID NO: 11 - DNA, 20 bp, Artificial sequence
  Synthetic oligonucleotide
tccatgacgttcctgacgtt

SEQ ID NO: 12 - DNA, 24 bp, Artificial sequence
  Synthetic oligonucleotide
tcgtcgttttgtcgttttgtcgtt

SEQ ID NO: 13 - DNA, 22 bp, Artificial sequence
  Synthetic oligonucleotide
tcgtcgttgtcgttttgtcgtt

SEQ ID NO: 14 - DNA, 29 bp, Artificial sequence
  Synthetic oligonucleotide
tcgcgaacgttcgccgcgttcgaacgcgg

SEQ ID NO: 15 - DNA, 22 bp, Artificial sequence
  Synthetic oligonucleotide
tcgtcgttttcggcgcgcgccg

SEQ ID NO: 16 - DNA, 25 bp, Artificial sequence
  Synthetic oligonucleotide
tcgtcgtcgttcgaacgacgttgat